(19) United States
(12) Patent Application Publication
(10) Pub. No.: US 2005/0214279 A1
(43) Pub. Date: Sep. 29, 2005

(54) Methods for stimulating human leukocytes to kill bacteria, yeast and fungi in biofilms that have formed in/on prosthetic devices, catheters, tissues and organs in vivo

(76) Inventors: Samuel C. Silverstein, New York, NY (US); Jeff Leid, Flagstaff, AZ (US); Yongmei Li, Philadelphia, PA (US); John D. Loike, Jamaica, NY (US); J. William Costerton, Marina del Rey, CA (US); Mark Shirtliff, Columbia, MD (US)

Correspondence Address: Leslie Gladstone Restaino;Brown Raysman Millstein Felder & Steiner LLP, 163 Madison Avenue, P.O. Box 1989, Morristown, NJ, 07962-1989, US

(21) Appl. No.: 10/943,700

(22) Filed: Sep. 17, 2004

Related U.S. Application Data
(60) Provisional application No. 60/504,068, filed on Sep. 19, 2003.

(51) Int. Cl. G01N 33/554; G01N 33/569; A61K 39/395; A61K 39/40

(52) U.S. Cl. 424/141.1; 424/164.1; 702/19; 435/7.32

(57) ABSTRACT

The present invention provides a method for stimulating human leukocytes to kill microorganisms in biofilms. The invention also provides a methods, compositions and kits for treating or preventing a biofilm infection in a mammal comprising administering a therapeutically effective amount of a complement protein and one or more antibodies which bind to a bacterial, yeast, fungal, carbohydrate or lipid epitope present in the biofilm. Additionally, the invention provides methods, compositions and kits for treating biofilm infection in a mammal which comprises administering to the mammal a therapeutically effective amount of a complement protein and a conjugate composition. The invention also provides methods for determining Critical Neutrophil Concentration and Neutrophil Extraction Efficiency in a mammal.

### RELATED APPLICATIONS

This application claims the benefit of U.S. Provisional Application Ser. No. 60/504,068, filed on Sep. 19, 2003, and entitled “Methods for Stimulating Human Leukocytes to Kill Bacteria,” the contents of which are hereby incorporated by reference herein.

### FIELD OF THE INVENTION

The present invention relates to a method for stimulating human leukocytes to kill microorganisms in biofilms. More particularly, the invention relates to a method and composition for treating and preventing biofilm infection by stimulating leukocytes to kill bacteria, yeast and fungi in biofilms.

### BACKGROUND OF THE INVENTION

A biofilm is a complex community of bacterial and other microbes adhering to an inert or living surface. In the last decade it has become evident that specific environmental conditions stimulate most bacteria to form structures called biofilms. Costerton, J. W., Stewart, P. S. & Greenberg, E. P. Bacterial biofilms: a common cause of persistent infections. Science 284, 1318-22. (1999); Stoodley, P., Sauer, K., Davies, D. G. & Costerton, J. W. Biofilms as complex differentiated communities. Annu Rev Microbiol 56, 187-209 (2002); Davies, D. G., Parsek, M. R., Pearson, J. P., Iglewski, B. H., Costerton, J. W. & Greenberg, E. P. The involvement of cell-to-cell signals in the development of a bacterial biofilm. Science 280, 295-8. (1998). Bacteria in biofilms are in very close contact with one another. They secrete substances called quorum sensing factors that signal them to produce copious amounts of weakly immunogenic exo-polysaccharides, which coat the biofilm and block access of phagocytic leukocytes, such as neutrophils and monocytes, to the bacteria within it. Efforts to disrupt and digest biofilms with lysosomal enzymes from neutrophils have proved unsuccessful. Biofilms also protect bacteria in the biofilms from antibiotics and oxidants. Stewart, P. S. & Costerton, J. W. Antibiotic resistance of bacteria in biofilms. Lancet 358, 135-8. (2001); Jensen, E. T., Kharazmi, A., Hoiby, N. & Costerton, J. W. Some bacterial parameters influencing the neutrophil oxidative burst response to Pseudomonas aeruginosa biofilms. Apmis 100, 727-33. (1992).

Biofilm infections of indwelling devices such as prosthetic joints, heart valves, and catheters are among the most serious and difficult infections to eradicate. Often, the device must be removed to cure the infection. When the prosthesis is a joint or a heart valve, the effects of a biofilm infection can be devastating.

In view of the severity and magnitude of problems caused by biofilm infection, methods and compositions to effectively prevent and treat biofilm infections are needed.

Complement opsonization of planktonic Staphylococcus epidermidis is required for neutrophils to kill them, both in stirred suspensions and in fibrin gels. Li, Y., et al. A critical concentration of neutrophils is required for effective bacterial killing in suspension. Proc. Natl. Acad Sci. U.S.A., 2002. 99(12):8289-94. Furthermore, the release of C5a from the surface of bacteria facilitates neutrophil killing of S. epidermidis embedded in fibrin gels. In the case of bacteria embedded in and surrounded by biofilm exopolysaccharides, IgG and complement opsonization and C5a release may be necessary to attract neutrophils to biofilms and to stimulate neutrophils to biofilm bacteria. Indeed, Meluleni, et al., reported that complement and antibodies vs. biofilm exopolysaccharides were absolutely required for neutrophil killing of P. aeruginosa in 1-day-old biofilms. Mucoid Pseudomonas aeruginosa growing in a biofilm in vitro are killed by opsonic antibodies to the mucoid exopolysaccharide capsule but not by antibodies produced during chronic lung infection in cystic fibrosis patients. J Immunol 155, 2029-38. (1995). Moreover, they documented that enzymatic hydrolysis of these exopolysaccharides enhanced neutrophil killing of P. aeruginosa in 1-day-old biofilms, but only under conditions in which specific antibodies vs. P. aeruginosa exopolysaccharides were absent. The Meluleni, et al., study was limited to biofilms that were 1-day old. Because, the prior art, including Meluleni, et al., has not studied neutrophil interactions with biofilms that were more than 1-day old, there is a need to elucidate the interaction of neutrophils with more mature biofilms (i.e., greater than 1-day old). Accordingly, the present invention relates to experiments studying the interaction of neutrophils with 1-, 5, and 10-day old biofilms.

Meluleni, et al., used a stirred suspension assay to examine neutrophil-biofilm interactions. However, by definition, biofilms form on or in tissues, not in suspension. Accordingly, in contrast with Meluleni, et al., in order to provide a more tissue-like environment to study neutrophil-biofilm interactions, experiments with respect to the present invention were conducted using a fibrin gel system rather than a stirred suspension assay. Specifically, a fibrin gel was used to explore interactions of neutrophils with S. epidermidis biofilms formed under flow conditions. The S. epidermidis biofilms were then harvested 1 to 10 days after seeding. These fibrin gel studies show that complement and IgG deposition on S. epidermidis in biofilms decreased with increasing age of the biofilms. Confocal laser fluorescence microscopy of intact biofilms incubated with normal human serum showed discontinuous deposits of complement and IgG on the surface of the biofilms. Electron microscopy showed neutrophils adhered tightly to the surfaces of 10 day-old biofilms. Strikingly, neutrophils killed >98% of S. epidermidis contained in 5-day-old biofilms, albeit at an efficiency seven times less than found for killing of planktonic S. epidermidis in these gels.

Neutrophil bactericidal activity in stirred suspensions is described by the equation bₒ=bt·e^{-k Pt+gt}(Eq. 1), in which k is the rate constant for bacterial killing, p is the neutrophil concentration, t is time, and g is the rate constant for bacterial growth. Li, Y., et al., A critical concentration of neutrophils is required for effective bacterial killing in suspension. Proc. Natl. Acad Sci. U.S.A., 2002. 99(12):8289-94. g/k describes a parameter we have termed the critical neutrophil concentration (CNC), below which bacterial concentration increases and above which bacterial concentration decreases. The CNC in stirred suspensions containing 10³ to 10⁷ CFU (colony forming unit)/ml S. epidermidis is ˜4×10⁵ neutrophils/ml, a value close to the blood neutrophil concentration (5×10⁵ neutrophils/ml) known to predispose humans to bacterial sepsis.

While it is useful to know the value of the CNC in stirred suspensions, it would be even more useful to know its value in tissues. This is because it is a critical parameter that determines whether bacteria can be eradicate from tissue and thereby prevented from entering the blood. Eq. 1 was used, to determine the value of k for killing of S. epidermidis in fibrin gels in vitro, and of E. coli in rabbit dermis in vivo, and have used these values, and those for g, to determine the CNC required to block growth of these bacteria in these environments. Furthermore, using experimentally determined values for blood neutrophil concentration, blood flow through, and tissue neutrophil concentration in, E. coli-inoculated rabbit dermis, the percent of blood neutrophils perfusing E. coli-inoculated rabbit dermis that immigrate into it was determined. We report that increased blood flow and neutrophil extraction efficiency (NEE) both are required for neutrophils to reach the CNC in rabbit dermis within 1-2 hr of E. coli inoculation.

### SUMMARY OF THE INVENTION

The present invention generally provides a method and composition for preventing and treating biofilm infection. In one embodiment, the invention provides a method for treating a biofilm infection in a mammal comprising administering to the mammal a therapeutically effective amount of a composition comprising a complement protein and one or more antibodies which bind to a bacterial, yeast, fungal, carbohydrate or lipid epitope present in the biofilm.

In another embodiment, the invention provides a method for treating a biofilm infection in an animal comprising administering to the animal a therapeutically effective amount of a composition comprising a complement protein, and a conjugate composition, said conjugate composition comprising one or more antibodies which bind to a bacterial, yeast, fungal, carbohydrate or lipid epitope present in the biofilm covalently linked with a masked or active protein selected from the group consisting of chemoattractants, chemokines, cytokines, glycosidases or proteases. In a specific embodiment, the mammal is human. Among other bacterial biofilm infections, the invention specifically provides for treating an s. epidernidis biofilm infection. In specific embodiments, the protein of the conjugate composition can be either masked or active. In other specific embodiments, the biofilm is formed on an indwelling device, a prosthetic device, a catheter or a tissue. In yet another embodiment, the antibody is a human or humanized monoclonal antibody.

In another embodiment, the invention provides a composition for treating a biofilm infection comprising a complement protein and one or more antibodies which binds to a bacterial, yeast, fungal, carbohydrate or lipid epitope present in the biofilm.

In another embodiment, the invention provides a composition for treating a biofilm infection comprising a complement and a conjugate composition, said conjugate composition comprising an antibody which binds to a bacterial, yeast, fungal, carbohydrate or lipid epitope present in the biofilm covalently linked with a protein selected from the group consisting of chemoattractants, cytokines, glycosidases or proteases.

In another embodiment, the invention provides a kit for use in treating a biofilm infection comprising a complement protein and an antibody which binds to a bacterial, yeast, fungal, carbohydrate or lipid epitope present in the biofilm.

In still another embodiment, the invention provides a kit for use in treating a biofilm infection comprising a complement protein, and a conjugate composition, said conjugate composition comprising an antibody which binds to a bacterial, yeast, fungal, carbohydrate or lipid epitope present in the biofilm covalently linked with a plasma protein selected from the group consisting of chemoattractants, cytokines, glycosidases or proteases.

In one embodiment, the invention provides a system for analyzing host defense against pathogens. More specifically, the invention provides a method for precisely predicting the efficiency of killing of bacterial in blood and in tissues by phagocytic white blood cells.

In a specific embodiment, the invention provides a method for determining critical neutrophil concentration (CNC) in a pathogen infected tissue comprising determining the concentration of neutrophils accumulated in a volume of the tissue for a period of time after initial infection (NC); determining the growth of the pathogen in the volume of tissue for the period of time after initial infection (PG); calculating the CNC on the basis of the parameters NC and PG by developing an algorithm of determining CNC as a function of NC and PG and applying the values of NC and PG of the tissue under examination to the algorithm.

In another embodiment, the invention provides a method for determining neutrophil extraction efficiency of a pathogen infected tissue comprising determining the concentration of neutrophils accumulated in a volume of the tissue for a period of time after initial infection (NC); determining the total number of neutrophils delivered to the volume of the tissue for the period of time after initial infection (NN); calculating the NEE on the basis of the parameters NC and NN by developing an algorithm of determining NEE as a function of NC and NN and applying the values of NC and NN of the tissue under examination to the algorithm.

Additional aspects of the present invention will be apparent in view of the description that follows.

### BRIEF DESCRIPTION OF THE FIGURES

The invention is illustrated in the figures of the accompanying drawings which are meant to be exemplary and not limiting, in which like references are intended to refer to like or corresponding parts, and in which:

FIG. 1A depicts C3 staining (with Syto-13 green) on the surface of 10-day-old S. epidermidis biofilm.

FIG. 1B depicts C3 staining (with Syto-13 green) of the middle of 10-day-old S. epidermidis biofilm.

FIG. 2A depicts an electron micrograph (magnification 3000×) of a portion of a fibrin gel containing pieces of 10-day-old S. epidermidis biofilm.

FIG. 2B depicts an electron micrograph (magnification 8000×) of a portion of a fibrin gel containing pieces of 10-day-old S. epidernidis biofilm.

FIG. 2C depicts an electron micrograph (magnification 6000×) of S. epidermidis in biofilms at time zero.

FIG. 3A depicts quadrant distribution of S. epidermidis that were single positive for C3 (lower right quadrant), single positive for IgG (upper left quadrant), double positives (upper right quadrant), and double negatives (lower left quadrant).

FIG. 3B depicts the fraction of bacteria opsonized with C3, IgG or both.

FIG. 4A depicts fluorescent intensity of C3 or IgG staining on planktonic bacteria (thin line) and biofilm bacteria (thick line).

FIG. 4B depicts fluorescent intensity of C3/IgG staining on biofilm bacteria relative to that of their respective controls.

FIG. 5A depicts the linear correlation of the fluorescence of BCECF-labeled S. epidermidis biofilms with the optical density of planktonic bacteria isolated from biofilms.

FIG. 5B depicts the linear correlation of the fluorescence of BCECF-labeled S. epidermidis biofilms with the number of viable bacteria.

FIG. 6 depicts neutrophil killing of S. epidermidis in 5-day-old biofilms.

FIG. 7 depicts cyto- and histo-grams of S. epidermidis from biofilms.

FIG. 8A depicts CFU of S. epidermidis recovered from fibrin gels containing neutrophils, normal human serum and these bacteria, at time zero or after 90 min. incubation at 37° C.

FIG. 8B depicts Bacteria killed=[1-b₉₀ₘᵢₙ(with neutrophils)/b₉₀ₘᵢₙ(bacterial alone)]×100%.

FIG. 8C depicts the mean S. epidermidis concentration (ordinate) recovered from fibrin gels containing the indicated initial concentrations of bacteria, neutrophils (abscissa) and normal human serum after 90 min. incubation.

FIG. 9A depicts confocal fluorescence micrographs of fibrin gels containing the indicated concentrations of Syto-13-stained neutrophils.

FIG. 9B depicts distances (sum)±SD measured between neutrophils in the fibrin gels shown in FIG. 9A.

FIG. 10A depicts concentrations of E. coli/ml dermis of normal and neutropenic rabbits calculated using data from Movat, H. Z., et al. Acute inflammation in gram-negative infection: endotoxin, interleukin 1, tumor necrosis factor, and neutrophils. Fed. Proc. 46, 97-104 (1987).

FIG. 10B depicts concentrations of neutrophils/ml dermis of normal and neutropenic rabbits calculated using data from Movat, H. Z., et al. Acute inflammation in gram-negative infection: endotoxin, interleukin 1, tumor necrosis factor, and neutrophils. Fed Proc 46, 97-104 (1987). Monocyte data are from Issekutz, T. B., Issekutz, et al. The in vivo quantitation and kinetics of monocytes migration into acute inflammatory tissue. Am. J. Pathol. 103, 47-55 (1981).

FIG. 10C depicts the effect of intra-dermal E. coli inoculation on blood flow/ml dermis (re-plotted from Kopaniak, M. M. and Movat, H. Z., Kinetics of acute inflammation induced by Escherichia coli in rabbits. II. The effect of hyperimmunication, complement depletion, and depletion of leukocytes. Am. J. Pathol. 110, 13-29 (1983)) and on neutrophil extraction efficiency (calculated as described in Methods).

### DETAILED DESCRIPTION OF THE INVENTION

In the following description of specific embodiments, reference is made to the accompanying drawings that form a part hereof, and in which is shown by way of illustration a specific embodiment in which the invention may be practiced. It is to be understood that other embodiments may be utilized and structural changes may be made without departing from the scope of the present invention.

Biofilm Infection

The present invention encompasses methods and compositions for use in preventing and treating biofilm infection in a subject. The methods and compositions generally stimulate human leukocytes to kill bacteria, yeast and fungi in biofilms that have formed in or on prosthetic devices, catheters, tissues and organs in vitro. The subject may be any mammal, but is preferably human.

The invention is based at least in part on the surprising discovery that neutrophils can kill approximately 97% of bacteria in relatively mature (e.g., 5-day old) biofilms. Specifically, neutrophils incubated at 37° C. in fibrin gels containing 40% human serum with fragments >1 mm³ of S. epidermidis biofilms kill approximately 97% of the S. epidermidis in these biofilms. Surprisingly, the mode of killing does not require phagocytosis of the bacteria. Rather, neutrophil adhere tightly to the biofilms and secrete products that kill the S. epidermidis.

Evidence suggests that anti-staphylococcal IgG and complement, present in normal human serum, bind to both the bacteria in the biofilm and to the surface of the biofilm; that complement component C3 becomes fixed to some of the bacteria in the biofilm via the alternate and classical pathways of complement activation; and that C3a and C5a are produced and released from the biofilm into the surrounding fibrin gel. Neutrophils are attracted to these biofilms by this C3a and C5a, and perhaps by other substances produced by the bacteria and/or by their interactions with human serum. The net result, is that neutrophils are attracted to the biofilms and adhere tightly to the surfaces of the biofilm via interactions of their Fc receptors, complement receptors, β-integrins (especially β₁ and β₂ integrins), and by lectin-like receptors with IgG, complement, fibronectin, and complex polysaccharides on the surfaces of the biofilm and the bacteria. These ligand receptor interactions stimulate neutrophils and monocytes to secrete the contents of their granuales onto the surfaces of the biofilm, and to produce H₂O₂, O₂, HOCl, NO, leukotrienes, chemokines (e.g., IL-8), cytokines (e.g., IL-1, TNFα), proteases and glycosidases, and other substances that may be toxic or cytolitic to the bacteria. As a consequence of these events, the bacteria and other microbes in the biofilm are killed. Bacteria and other microbes that escape from the biofilm are phagocytosed and killed by the neutrophils. Therefore, by linking active or masked chemoattractants (e.g., C5a, IL-8), cytokines (e.g., G-CSF, IL-12), and glycosidases and proteases to antibodies directed against one or more of the surface polysaccharides, proteins, or lipids expressed by the biofihm, antibodies can be created that will bind to the biofilms and to the bacteria within it that will, in combination with complement, promote migration and adhesion of neutrophils, monocytes, eosinophils, basophils, and/or NK cells to biofilms and stimulate these leukocytes to adhere to and secrete substances that will kill both the microbes in the biofilm and planktonic microbes in the surrounding environment.

Accordingly, in one embodiment, the invention provides a method for treating biofilm infection in a mammal which comprises administering a therapeutically effective amount of a composition comprising a complement protein and one or more antibodies which bind to a bacterial, yeast, fungal, carbohydrate or lipid epitope present in the biofilm. The term “therapeutically effective amount,” as used herein means the quantity of the composition according to the invention which is necessary to prevent, cure, ameliorate or at least minimize the clinical impairment, symptoms or complications associated with biofilm infection. As used in the present invention “complement protein” refers to the large number of enzymes, proenzymes, and other proteins which form the principle effector mechanism of immunity in extracellular body fluids. Examples of complement protein within the scope of the invention include, but are not limited to, C1-C9 and Factors B, D, H, I, P.

Another embodiment of the invention provides a method for treating biofilm infection in a mammal which comprises administering to the mammal a therapeutically effective amount of a composition comprising complement protein and a conjugate composition. The conjugate composition comprises one or more antibodies which bind to a bacterial, yeast, fungal, carbohydrate or lipid epitope present in the biofilm, which is covalently linked to a protein selected from the group consisting of chemoattractants, chemokines, cytokines, glycosidases or proteases. As used in the present invention, “conjugate composition” refers to the composition comprising an antibody directed to an epitope in the biofilm covalently linked with a chemoattractant, chemokine, cytokine, glycosidase or protease.

The protein linked to the antibody of the conjugate composition can be either masked or unmasked (active). Techniques for conjugating therapeutic moieties to antibodies are well known, e.g., Thorpe, et al., the preparation and cytotoxic properties of antibody-toxin conjugates, Immunol. Rev., 62:119-58 (1982); Arnon, et al., “Monoclonal Antibodies For Immunotargeting Of Drugs In Cancer Therapy,” in Monoclonal Antibodies And Cancer Therapy, Reisfeld, et al., (eds.), pp. 243-56 (Alan R. Liss, Inc. 1985) (incorporated herein by reference).

Antibodies of the present invention refer to immunoglobulin molecules and immunologically active portions of immunoglobulin molecules, i.e., molecules that contain an antigen binding site which binds to an epitope present in a biofilm. The epitope may be a bacterial, yeast, fungal, carbohydrate or lipid epitope. The immunoglobulin molecules of the present invention can be of any type including, but not limited to, IgG, IgE, IgM, IgD, F(ab)′, F(ab)₂ and IgA. In a specific embodiment, the antibody used is a monoclonal antibody. In accordance with the present invention, monoclonal antibodies can be prepared using a wide variety of techniques known in the art including, but not limited to, the use of hybridoma, recombinant, and phage display technologies, or a combination thereof. For example, monoclonal antibodies can be produced using hybridoma techniques including those known in the art and taught, for example, in Kohler and Milstein, (1975, Nature 256:495-497; and U.S. Pat. No. 4,376,110), the human B-cell hybridoma technique (Kosbor, et al., 1983, Immunology Today 4:72; Cole, et al., 1983, Proc. Natl. Acad. Sci. USA 80:2026-2030), and the EBV-hybridoma technique (Cole, et al., 1985, Monoclonal Antibodies And Cancer Therapy, Alan R. Liss, Inc., pp. 77-96; Harlow, et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (incorporated herein in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. In a preferred embodiment, the monoclonal antibody is a humanized antibody or a human antibody.

It will be appreciated by those of skill in the art that in accordance with the present invention, that vaccines comprising an antigen or antigens from a biofilm can also readily be made.

In one embodiment of the invention, the biofilm to be treated is formed on an indwelling device. As used herein, “indwelling device” refers to any device left within the body for an extended period of time such as a catheter or prosthesis. In a specific embodiment, the biofilm is formed on a prosthetic device. In another embodiment the biofilm is formed on a catheter. In yet another embodiment, the biofilm is formed on tissue. It will be appreciated by those of skill in the art that in accordance with the present invention, the therapeutic composition of the present invention can be infused or otherwise delivered into any fluid, tissue or structure of the body including but not limited to the blood, tissues, cerebral spinal fluid (CFS), eye, oral cavity, peritoneum, pleural spaces, and/or joints of patients infected with biofilm-forming bacterium.

In another aspect, the invention provides a method of preventing a biofilm infection in a mammal which comprises administering to the mammal a therapeutically effective amount of a composition comprising a complement protein and one or more antibodies which bind to a bacterial, yeast, fungal, carbohydrate or lipid epitope present in the biofilm.

In yet another embodiment, the invention provides method for preventing a biofilm infection in an mammal comprising administering to the mammal a therapeutically effective amount of a composition comprising a complement protein and a conjugate composition, the conjugate composition comprising one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases. The protein linked to the antibody of the conjugate composition can be either masked or unmasked (active).

As used herein, “preventing biofilm infection” includes preventing the initiation of a biofilm infection, delaying the initiation of a biofilm infection, preventing the progression or advancement of a biofilm infection, slowing the progression or advancement of a biofilm infection, and delaying the progression or advancement of a biofilm infection.

Another embodiment of the invention provides compositions for treating or preventing biofilm infection. The composition comprises a complement protein and one or more antibodies which bind to a bacterial, yeast, fungal, carbohydrate or lipid epitope present in the biofilm. A composition for treating a biofilm infection is also provided which comprises a complement protein and a conjugate composition, said conjugate composition comprising: one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases. The protein of the conjugate composition can be either masked or unmasked (active). As discussed above, the composition of the present invention can be infused or otherwise delivered into any fluid, tissue or structure of the body, including but not limited to the blood, tissues, cerebral spinal fluid (CFS), eye, oral cavity, peritoneum, pleural spaces, and/or joints of patients infected with biofilm-forming bacterium. The protein linked to the antibody of the conjugate composition can be either masked or unmasked (active).

Another embodiment of the invention provides a kit for use in treating a biofilm infection comprising a complement protein and an antibody which binds to a bacterial, yeast, fungal, carbohydrate or lipid epitope present in the biofilm. A kit for use in treating a biofilm infection is also provided which comprises a complement protein, and a conjugate composition, said conjugate composition comprising one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases.

Critical Neutrophil Concentration

Host defense against bacterial infection requires an adequate concentration of neutrophils in tissues. However, the precise relationship between blood neutrophil concentration, tissue neutrophil concentration, and neutrophil bactericidal activity in tissues has been previously unknown. Accordingly, fibrin gels, which provide a tissue-like environment, were used to study neutrophil bactericidal activity in a tissue-like environment. The present invention is based, at least in part, on the discovery that killing of Staphylococcus epidermidis by neutrophils in these fibrin gels is described by a single exponential equation that combines neutrophil bacterial killing rate and bacterial growth rate. Data on neutrophil bactericidal activity in fibrin gels and rabbit dermis was used to solve this equation for the bacterial killing rate constant, and used the value of this constant to determine the critical neutrophil concentration, the neutrophil concentration at which the bacterial killing rate equals the bacterial growth rate, required to block bacterial growth. The critical neutrophil concentration was 4×10⁶ neutrophils/ml fibrin gel and 7.7×10⁶ neutrophils/ml dermis, 10 and 19-fold higher, respectively, than in stirred suspensions. These results provide the first quantitative evidence that tissue neutrophil concentration is the limiting factor that predisposes neutropenic patients to sepsis.

Accordingly, the invention provides a system for analyzing host defense against pathogens. More specifically, the invention provides a method for precisely predicting the efficiency of killing of bacteria in blood and in tissues by phagocytic white blood cells. In a specific embodiment, the invention provides a method for determining critical neutrophil concentration (CNC) in a pathogen infected tissue comprising determining the concentration of neutrophils accumulated in a volume of the tissue for a period of time after initial infection (NC); determining the growth of the pathogen in the volume of tissue for the period of time after initial infection (PG); calculating the CNC on the basis of the parameters NC and PG by developing an algorithm of determining CNC as a function of NC and PG and applying the values of NC and PG of the tissue under examination to the algorithm.

In another embodiment, the invention provides a method for determining neutrophil extraction efficiency of a pathogen infected tissue comprising determining the concentration of neutrophils accumulated in a volume of the tissue for a period of time after initial infection (NC); determining the total number of neutrophils delivered to the volume of the tissue for the period of time after initial infection (NN); calculating the NEE on the basis of the parameters NC and NN by developing an algorithm of determining NEE as a function of NC and NN and applying the values of NC and NN of the tissue under examination to the algorithm.

As used herein, the term “critical neutrophil concentration” refers to the neutrophil concentration which prevents or blocks bacterial growth. As used in the present invention, the term “neutrophil extraction efficiency” describes the number of neutrophils that enter infected tissue divided by the number of neutrophils in the blood perfusing the same tissue.

The present invention is described in the following Examples, which are set forth to aid in the understanding of the invention, and should not be construed to limit in any way the scope of the invention as defined in the claims which follow thereafter.

### EXAMPLES

Detailed Description of Figures

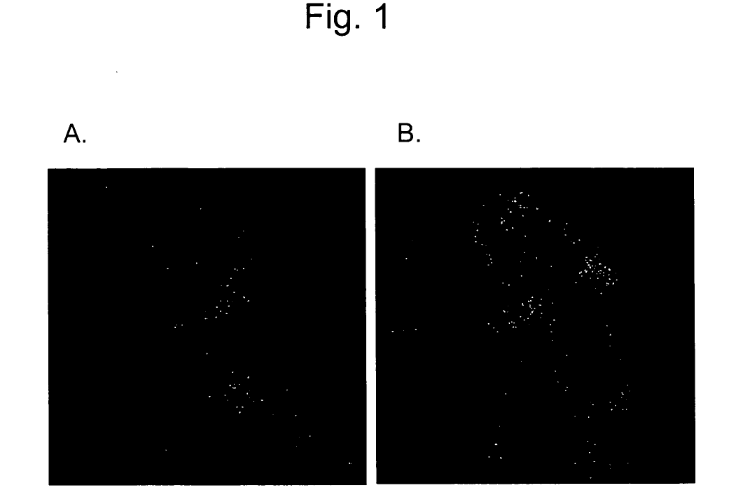

FIG. 1 shows C3 staining on the surface (A) and middle (B) of a 10-day-old S. epidermidis biofilm. Images were obtained by confocal laser scanning microscopy. Shown are 0.5 μm thick optical section of the surface and middle of 10-day-old biofilm opsonized in normal human serum, incubated with goat anti-human C3, washed, and then incubated with rhodamine-conjugated donkey anti-goat IgG (C3, red). S. epidermidis were stained green by Syto-13 (green). The sections were 6 μm away from each other. Magnification 100×.

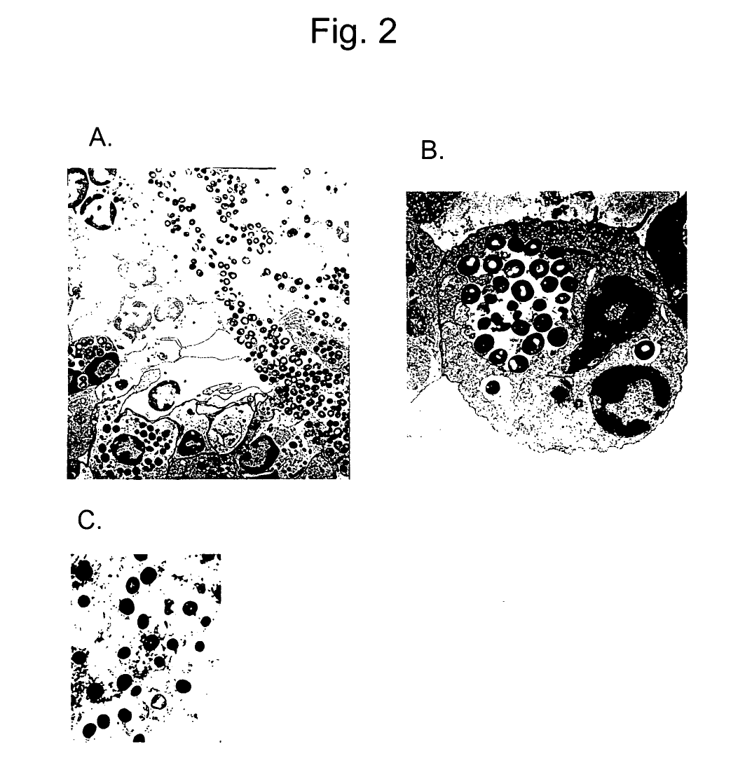

FIG. 2 depicts Electron micrographs of portions of three different fibrin gels containing pieces of 10-day-old S. epidermidis biofilms. Human neutrophils formed tight adhesion with biofilms and ingested S. epidermidis in biofilms after 6 hr incubation at 37° C. (A. Magnification 3000×; B. Magnification 8000×.). S. epidermidis in biofilms at time zero showing electron dense center. (C. Magnification 6000).

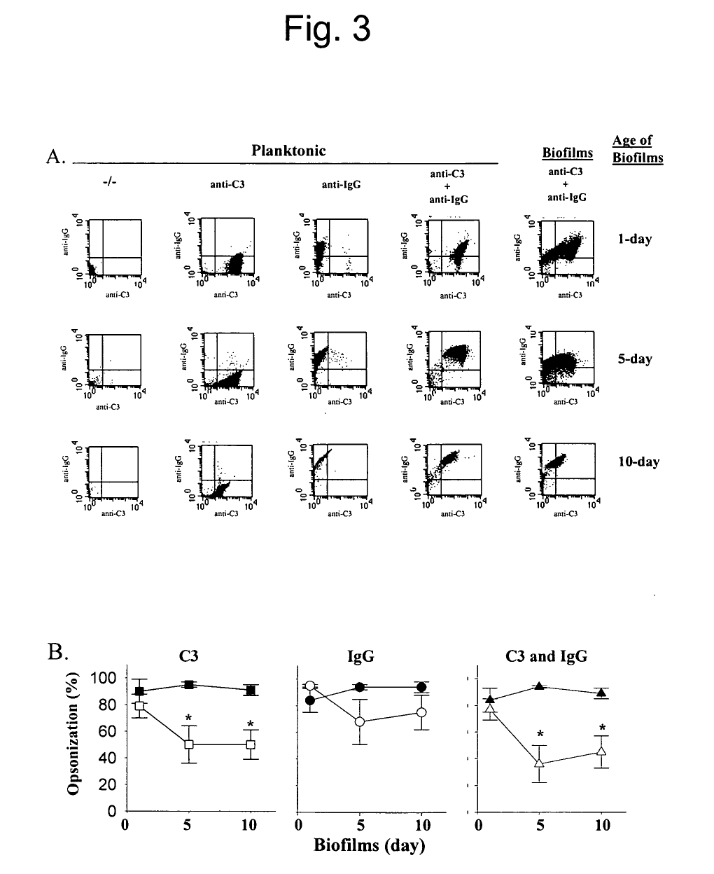

FIG. 3 shows the fraction of S. epidermidis in 1-, 5-, 10-day-old biofilms that were opsonized with C3 and/or IgG. S. epidermidis released from biofilms by sonication (planktonic) or whole pieces of biofilms were opsonized in normal serum (biofilm), sonicated again, and analyzed for C3/IgG deposition by flow cytometry. 10,000 events were analyzed, and quadrant positions were defined using singly stained planktonic bacteria. (A) Result from a representative experiment showing quadrant distribution of S. epidermidis that were single positive for C3 (lower right quadrant), single positive for IgG (upper left quadrant, double positives (upper right quadrant), and double negatives (lower left quadrant). (B) Fraction of bacteria opsonized with C3, IgG or both. Open symbols were bacteria opsonized in normal serum while embedded in biofilms; solid symbols were controls. Data represent the mean and SEM of 5 independent experiments for 10-day-old biofilms, three independent experiments for 5-day-old biofilms, and 2 independent experiments for 1-day-old biofilms. *, p<0.05 by paired Student's t test.

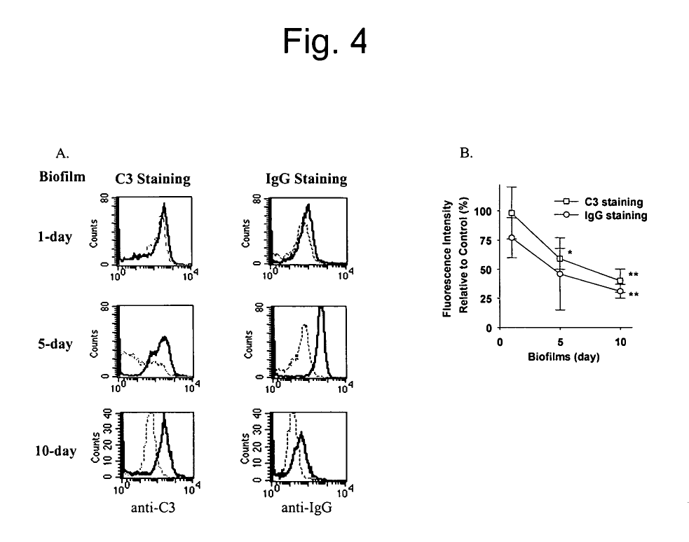

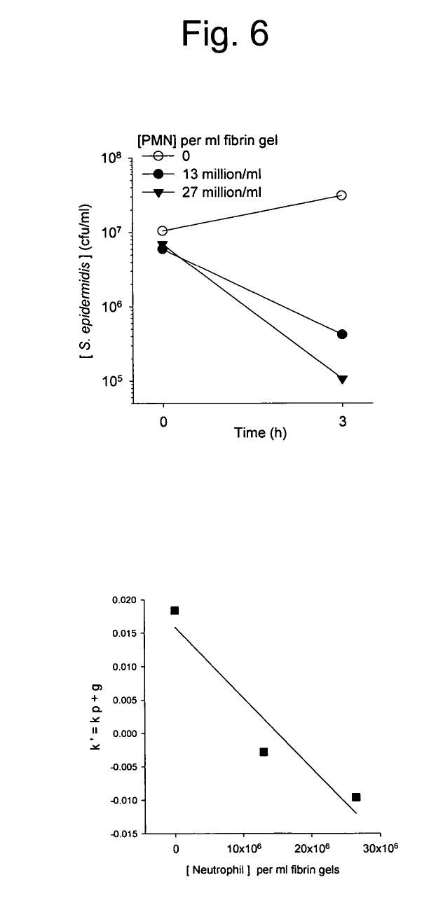

FIG. 4 depicts the relative amounts of C3 and/or of IgG deposited on S. epidermidis in 1-, 5-, and 10-day-old biofilms. S. epidermidis released from biofilms by sonication (planktonic) or whole pieces of biofilms were opsonized in normal serum (biofilm), sonicated again, and analyzed for C3/IgG deposition by flow cytometry as described in FIG. 6-3. (A) Fluorescent intensity of C3 or IgG staining on planktonic bacteria (thin line) and biofilm bacteria (thick line). (B) Fluorescent intensity of C3/IgG staining on biofilm bacteria relative to that of their respective controls, calculated as described in Methods.

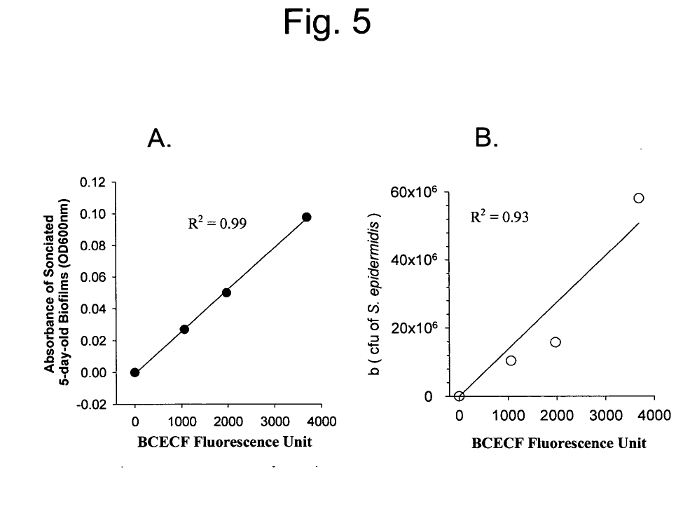

FIG. 5 shows fluorescence of BCECF-labeled S. epidermidis biofilms correlates linearly with (A) the optical density of planktonic bacteria isolated from biofilms and (B) the number of viable bacteria. Five-day-old S. epidermidis biofilms were incubated in PBS-GHSA containing 6 mM BCECF-AM for 30 min at 37° C. and washed. Serial dilutions of BCECF-labeled biofilms were measured for fluorescence at Ex 490 nm/Em 530 nm, and then sonicated to release bacteria. Suspensions of the released bacteria were measured for absorbance at 600 nm, serially diluted, and plated out as described in Methods. Shown results of a representative experiment and the functions fitted to data.

FIG. 6 depicts neutrophil killing of S. epidermidis in 5-day-old biofilms. Fibrin gels (300 ml in volume) containing BCECF-labeled 5-day-old S. epidermidis biofilms, 40% normal human serum, and the indicated concentrations of human neutrophils were incubated for 3 h at 37° C. The number of viable bacteria in biofilms embedded in fibrin gels at time zero and after a 3 h incubation were determined by referring to a standard curve of BCECF-fluorescence or by pour-plate method, respectively, as described in Methods. k′ and k were obtained as described in Methods. Shown are results from one of two independent experiments.

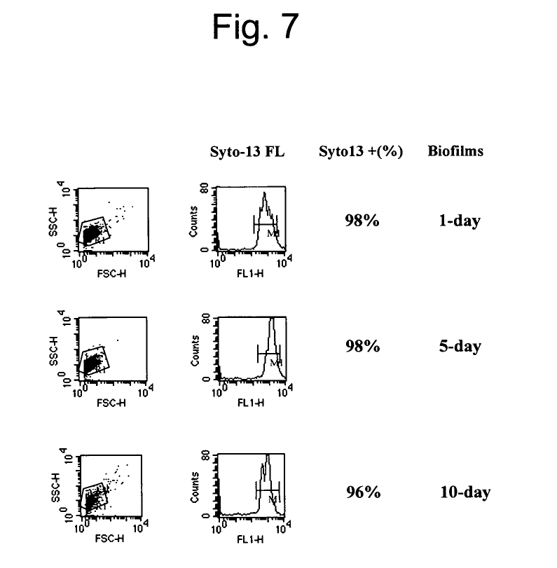

FIG. 7 shows cyto- and histo-grams of S. epidermidis from biofilms. Planktonic S. epidermidis, obtained by sonication of biofilms, was stained with Syto-13, and analyzed by flow cytometry. 10,000 events were collected for each analysis. Shown are distributions of events on a dot plot of FSC/SSC, the Syto-13 staining of the gated population (>95% total events), and the percentage of gated population positively stained.

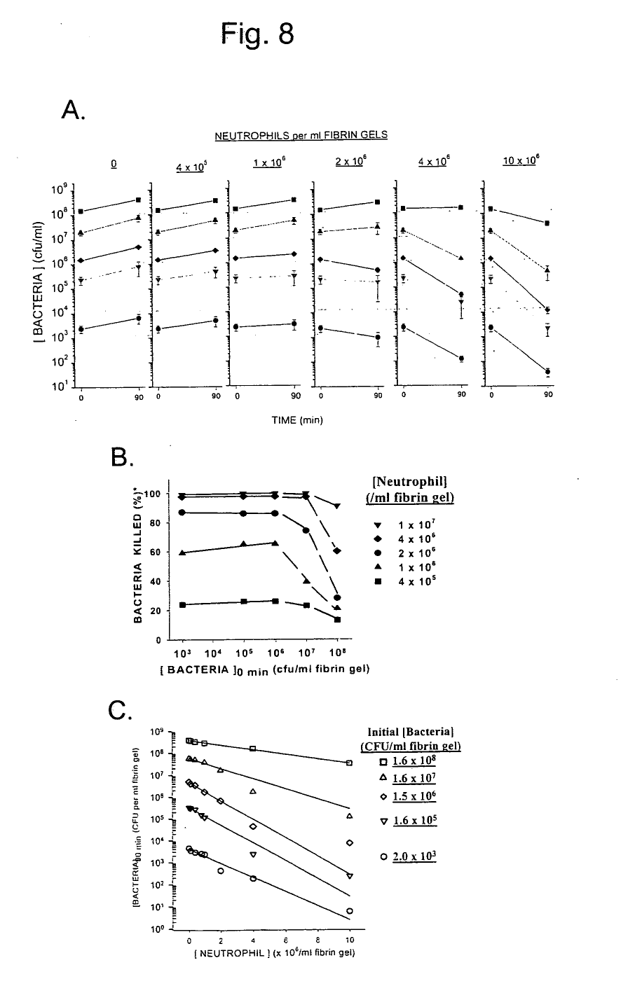

FIG. 8 shows that neutrophil concentration determines the number of S. epidermidis remaining viable after co-incubation in fibrin gels. a. CFU of S. epidermidis recovered from fibrin gels containing neutrophils, normal human serum and these bacteria, at time zero or after 90 min incubation at 37° C. Each data point represents the mean and SEM from five independent experiments, each performed in duplicate. b, Bacteria killed=[1-b₉₀ₘᵢₙ(withneutrophils)/b₉₀ₘᵢₙ(bacteria alone)]×100%. c, Symbols are the mean S. epidermidis concentration (ordinate) recovered from fibrin gels containing the indicated initial concentrations of bacteria, neutrophils (abscissa), and normal human serum after 90 min incubation. Lines are functions fitted to the data by non-linear regression analyses with Eq. 1. R² for each line is >0.98. Shown are results from one experiment representative of four experiments performed for each bacterial inoculum. Results of regression analyses of all experiments are summarized in Table 1.

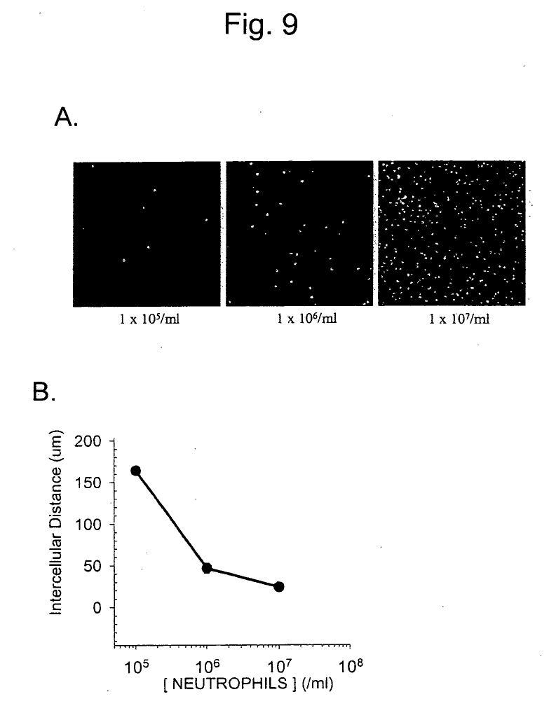

FIG. 9 shows that neutrophils are uniformly distributed in fibrin gels. a, Confocal fluorescence micrographs of fibrin gels containing the indicated concentrations of Syto-13-stained neutrophils. b, Distances (μm)±SD measured between neutrophils in fibrin gels shown in a.

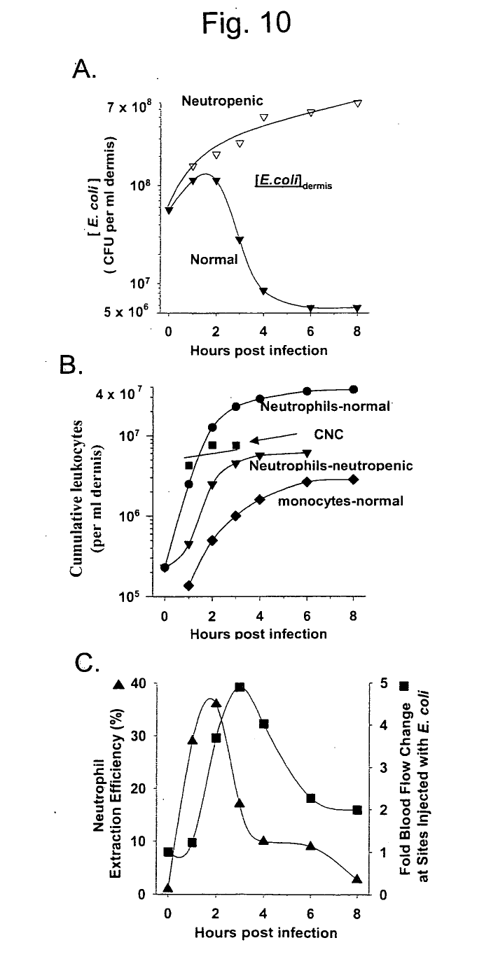

FIG. 10 depicts neutrophil, monocyte, and E. coli concentrations, and blood flow, per ml E. coli-inoculated rabbit dermis. a, Concentrations of E. coli/ml dermis of normal and neutropenic rabbits were calculated using data from Movat, H. Z., et al. Acute inflammation in gram-negative infection: endotoxin, interleukin 1, tumor necrosis factor, and neutrophils. Fed. Proc. 46, 97-104 (1987). b, Concentrations of neutrophils/ml dermis of normal and neutropenic rabbits were calculated using data from Movat, H. Z., et al. Acute inflammation in gram-negative infection: endotoxin, interleukin 1, tumor necrosis factor, and neutrophils. Fed. Proc. 46, 97-104 (1987). Monocyte data are from Issekutz, T. B., Issekutz, et al. The in vivo quantitation and kinetics of monocytes migration into acute inflammatory tissue. Am. J. Pathol. 103, 47-55 (1981). c, Effect of intra-dermal E. coli inoculation on blood flow/ml dermis (re-plotted from Kopaniak, M. M. and Movat, H. Z., Kinetics of acute inflammation induced by Escherichia coli in rabbits. II. The effect of hyperimmunication, complement depletion, and depletion of leukocytes. Am. J. Pathol. 110, 13-29 (1983)) and on neutrophil extraction efficiency (calculated as described in Methods).

Biofilm Infection

Materials and Methods

Antibodies

FITC-conjugated F(ab′)2 of goat anti-human C3 was from Protos Immunoresearch (Burlingame, Calif.); Phycoerythrin (PE)-conjugated F(ab′)2 of goat anti-human IgG (H+L chains) was from Jackson ImmunoResearch Laboratories (West Grove, Pa.). Unlabeled goat anti-human C3 IgG was from Sigma (Saint Louis, Mich.); unlabeled mouse anti-human IgG (H+L chains), was from Pierce (Rockford, Ill.). F(ab′)2 of rhodamine-labeled goat anti-mouse IgG and rhodamine-labeled donkey anti-goat IgG were from Molecular Probes (Eugene, Oreg.).

Biofilm

S. epidermidis biofilms grown for 1, 5, or 10 days under shear stress, were prepared by Drs. Jeff Leid, Mark Shirtliff and William Costerton (Montana State University, Bozeman, Mont.), shipped in an iced container overnight to Columbia University, and were used for experiments immediately on the day of arrival and the one after. Before experiments, biofilms were placed on cell strainers and washed gently with 50 ml PBS (Dulbecco's PBS with Ca++ and Mg++).

Confocal Laser Scanning Microscopy

C3 and IgG deposition in and on biofilms was examined using a Carl-Zeiss LSM one photon inverted confocal laser scanning microscope. To reduce non-specific binding of antibody, pieces of biofilm that had been incubated at 37° C. for 30 min with normal human serum were rinsed on cell strainers with PD-BSA, and then incubated for 15 min at room temperature in 1 ml of PD-BSA containing 2.6 μg/ml of goat-anti-human C3, or with 1 ml buffer containing 2.6 μg/ml of mouse-anti-human IgG. To visualize the primary antibodies bound to their cognate antigens in/on biofilms, the biofilms were washed three times in PD-BSA and incubated for 15 min at 4° C. in 1 ml PD-BSA containing 2 μg/ml of the respective rhodamine-labeled secondary antibodies. The biofilms then were washed in PD-BSA, and the bacteria they contained were stained with 5 μM Syto-13® nucleic acid stain (Molecular Probes). Immunofluoresent images were obtained using Carl Zeiss LSM 410 under 100×oil immersion lens. As negative controls, biofilms that were not incubated in serum were similarly stained with the primary antibodies, secondary antibodies and Syto-13®. They showed no unspecific bindings of the antibodies.

Transmission Electron Microscopy

Biofilms (10-day-old) were washed three times in PBS-GHAS PBS with Ca⁺⁺, Mg⁺⁺, glucose and human serum). Fibrin gels (40 μl in volume) containing 2 mg/ml fibrinogen, pieces of washed biofilms, 50% normal serum, were formed on tissue culture inserts. The top of each gel was added 50 μl PBS-GHSA containing human neutrophils (1.6×106), and the gels were incubated for 6 h 37° C. in a humidified incubator containing 5% CO2/95% air and then processed for transmission electron microscopy.

Sonication of Bioflims

For flow cytometric analyses, bacterial suspensions were prepared from biofilms by sonicafing the biofilms in PD-BSA at 4° C. with ˜30 pulses of a microprobe mounted on a sonicator (Ultrasonic, Plainview, N.Y.) set to 30% duty output and 3.5 output control. The viability of bacteria was not affected by up to 200 pluses of sonication at the above settings, determined in a preliminary experiment by comparing the colony forming units of an overnight culture of S. epidermidis before and after sonicafion (data not shown). Under light microscopy, the bacterial suspensions prepared from biofilms consisted mostly of single or double bacteria with occasional small clusters containing ˜20 bacteria (not shown).

Flow Cytometric Analysis

Flow cytometric analyses were performed on a FACScalibur equipped with a 488 nm argon laser (Becton Dickinson Immunocytometry Systems, San Jose, Calif.). Log parameters were used for FSC, SSC, FL1 and FL2. Data were acquired and analyzed with CellQuest software (Becton Dickinson Immunocytometry Systems, San Jose, Calif.).

FSC Setting

The FSC setting for detecting S. epidermidis was optimized using Syto-13-stained planktonic bacteria isolated from biofilms. The range of FSC for specifically detecting S. epidermidis was between E00/Amp gain 5 and E01 /Amp gain 2. PBS-BSA containing Syto-13 labeled, sonicated S. epidermidis (2×10⁸ CFU (colony forming unit)/ml) was compared to buffer containing Syto-13 alone. Within the specific setting indicated above, events of Syto-13-labeled S. epidermidis (2×10⁸ CFU/ml) were counting at ˜100-200/second with the flow rate set to High. No events were detected in PBS-BSA containing Syto-13 alone. With FSC below the setting, no events were detected in PBS-BSA containing 2×10⁸ CFU/ml S. epidermidis. When FSC was over the maximum detection limit for bacteria, such as at E03, even buffer gives 10,000 events. For flow cytometric analyses, >95% of events sampled from these bacterial suspensions clustered within a narrow range of FSC, indicating their similarity in size (FIG. 7). More than 96% of events were confirmed to be due to bacteria by the green fluorescence of Syto-13.

Compensation

Because of spectral overlap of FITC into the FL2 detector, compensation for FL2 (% FL1-FL2) of 13-26% was made using bacteria stained with either FITC-conjugated anti-C3 or PE-conjugated-anti-IgG.

Flow Cytometric Analysis of Biofilm Opsonization

Biofilms were incubated at 37° C. in PBS-GHSA containing 50% normal human serum for 30 min. They then were pelleted (with the supernatant saved for latter use), washed on cell strainers with 50 ml PBS to remove residual serum, and sonicated as described to yield homogenous suspensions of bacteria (Biofilms). The concentration of bacteria was determined by the absorbance of the bacterial suspensions at 600 nm (A600 nm), and reference to a curve relating A600 nm to CFU/ml of viable S. epidermidis. Using the bacterial concentration thus determined, a portion of the bacterial suspension was re-incubated with the serum-containing supernatant for 10 min at 37° C. at the same bacterial concentration as in biofilms. These bacteria served as fully opsonized controls (planktonic). Suspensions of S. epidermidis were washed in PD-BSA (Dulbecco's PBS without Ca++ and Mg++, supplemented with 2% BSA) and incubated at 2×10⁷ CFU/ml at 4° C. for 15 min with the indicated antibody(ies) (i.e., PE-conjugated goat anti-human IgG, 1:200 dilution; FITC-conjugated goat anti-human C3, 5 μg/ml). Bacteria were washed, resuspended in PD-BSA, briefly sonicated, and analyzed by flow cytometry using the FSC setting and compensation setting described above. 10,000 events were analyzed for each sample.

Analyses of Flow Cytometry Data

In a dot plot of FL1 (C3) versus FL2 (PE), S. epidermidis plotted in the upper right, lower right, upper left and lower left quadrant were double positives for C3 and IgG, single positive for C3, single positives for IgG, or double negatives, respectively. Quadrant statistics obtained with CellQuest Software were used for the following calculation:
1) Fraction of bacteria opsonized with C3 (%)=double positive (%)+single positive for C3 (%)
2) Fraction of bacteria opsonized with IgG (%)=double positive (%)+single positive for IgG (%)
3) Fraction of bacteria opsonized with C3 and IgG (%)=double positive (%).

Relative Fluorescent Intensity (%)

Relative fluorescent intensity (%) was calculated as follows:

(Mean Fluorescent Intensity of biofilm-associated bacteria/Mean Fluorescent Intensity of planktonic bacteria)×100%.

Bacterial Killing By Neutrophils In Stirred Suspensions

Biofilms were incubated in buffer containing the indicated concentration of normal human serum, washed three times to remove serum, and the bacteria contained in them released by sonication as described above. For bacterial killing, the suspension assay described in Chapter 3 was used. In brief, 500 μl PBS-G-HSA containing human neutrophils (4×10⁶/ml), S. epidermidis (˜1×10⁵ CFU/ml) from biofilms incubated with or without 10% normal human serum were placed in 1.5 ml sterile tubes. Where indicated, 10% normal human serum was added to the tubes. The tubes were incubated at 37° C. for 90 min on an orbital shaker rotating at 200 rpm. After 90 min, the number of viable bacteria in the mixtures was determined as described previously using pour-plate method. Biofilms were opsonized, washed three times to remove serum and broken up into homogenous bacterial suspensions as described above.

Standard Curves for the Relationship Between BCECF-fluorescence of S. epidermidis in Biofilms and Numbers of CFU of S. epidermidis in Biofllms

Biofilms were placed on 40 μm pore-size cell strainers, and washed with 50 ml PBS-GHSA to remove planktonic bacteria. Pieces of biofilm were re-suspended in 5 ml PBS-GHSA containing 6 μM BCECF-AM, incubated at 37° C. for 30 min, and rinsed on cell strainers with another 50 ml PBS-GHSA. 300 μl serial dilutions of the BCECF-labeled biofilms were placed in a 48-well place, and measured for fluorescence at Ex490 nm/Em 530 nm in a Cytoflour II. 700 μl PBS were added to each well to wash off the biofilms, the 1-ml suspensions were placed in 1.5 ml Eppendorf tubes, and sonicated to release bacteria, as described. The absorbance of the sonicated samples was measured at 600 nm, and used to approximate CFU/ml S. epidermidis in each sample by reference to a standard curve previously established for S. epidermidis H753 relating A600 nm of a suspension of S. epidermidis to the number of CFU/ml of S. epidermidis in the suspension. Samples then were serially diluted, plated on TSB nutrient agar, cultured overnight, and the number of in each sample was calculated from the colony counts. Similarly, a standard curve was developed relating the BCECF fluorescence of S. epidermidis in each biofilm sample to the number of CFU of S. epidermidis in that sample.

Neutrophil Killing of S. epidermidis in Biofilms Embedded in Fibrin Gels

Three-hundred μl PBS-GHSA containing BCECF-labeled biofilms, 40% normal human serum, with or without human neutrophils (13×10⁶/ml and 26×10⁶/ml), 1 mg/ml fibrinogen, and 0.3 U thrombin was added to a 48-well plate, incubated at room temperature for 5 min. 10 μl PPACK (10⁻⁷M) then was added to inhibit thrombin. The fibrin gels then were measured for fluorescence in a Cytofluor at Ex490 nm/Em530 nm, and incubated at 37° C. for 3 h. As a control for background fluorescence, fibrin gels containing serum alone or neutrophils and serum were similarly prepared and measured for fluorescence, which then was subtracted from the fluorescence of the gels containing neutrophils and BCECF-labeled biofilms. To determine the number of viable bacteria in fibrin gels, the gels were digested with 600 μPBS containing 5 mg/ml trypsin and 20 μM cytochalasin D for 15 min at 37° C. and the lysate was sonicated. The samples then were diluted 10×in pH 11 distilled water and incubated for 5 min to lyse neutrophils. Serial dilutions were made and plated on TSB agar. The plates were incubated overnight at 37° C., and the numbers of colonies were counted manually.

Calculation of k for Neutrophil Killing of S. epidermidis in 5-day-old Biofilms Embedded in Fibrin Gels

The initial inoculum (bo) and the number of viable bacteria remaining in fibrin gels determined as described above, were used to calculate k′ using Equation 3-5. The value of k was obtained by fitting Equation 3-6 to values of k′ on Sigma Plot.

General Materials and Methods

Thrombin, fMLP, carboxypeptidase Y, cytochalasin D, and Histopaque 1077 and 1119 were from Sigma (St. Louis, Mo.). PPACK (D-phenylanalyl-L-propyl-L-arginine chloromethyl ketone) and LTB4 were from Calbiochem-Novabiochem (San Diego, Calif.). Human fibrinogen was from American Diagnostica Inc. (Greenwich, Conn.). Cell culture inserts (0.4 μm pore size, 24-well plate format), tissue culture plates (24-well and 48-well format), agar, and Trypticase Soy Broth (TSB) were from Becton Dickinson (Franklin Lakes, N.J.). Heparin was from Elkins-Sinn Inc. (Cherry Hill, N.J.).

Staphylococcus epidermidis

S. epidermidis H753, a clinical isolate from the cerebrospinal fluid (CSF) of a patient with an infected CSF shunt, was provided by the Diagnostic Microbiology Laboratory at Columbia-Presbyterian Hospital. For experiments, 3% TSB was inoculated with S. epidermidis from a single colony and incubated with shaking overnight at 37° C. The overnight culture was sub-cultured into fresh TSB, grown to late log phase, pellet, washed three times in phosphate buffer saline (PBS) and re-suspended in PBS. The optical density (OD) of this suspension at 600 nm was monitored and colony forming units (CFU) of S. epidermidis were determined by reference to a standard curve relating OD at 600 nm to the CFU of S. epidermidis.

Human Sera

Normal human serum (NS) was prepared by incubating human AB plasma (New York Blood Center, New York, N.Y.) with 1 U/ml thrombin at room temperature for 15 min, and centrifuging the mixture at 8,000 g to remove fibrin. NS was then filter sterilized using 0.22 μm filters (Pall Corp., Ann Arbor, Mich.). Heat inactivated human serum (HIS) was prepared by heating NS at 56° C. for 30 min. Zymosan-activated NS (ZAS) was prepared as described 142. C5-deficient serum was from Sigma (St. Louis, Mo.) or provided by Dr. John P. Leddy (Allergy/Immunology & Rheumatology Clinical Group, Rochester, N.Y.). All sera were stored at −80° C. until use.

Human Neutrophils

Neutrophils were prepared as described. Briefly, fresh heparinized blood was obtained from healthy adult volunteers after informed consent. Neutrophils were isolated by centrifugation on Histopaque 1077 and 1119 gradients. Contaminating RBCs were removed by hypotonic lysis. The purity of neutrophils isolated by this method was >95%, as determined by Wright-Giemsa staining. Purified neutrophils were resuspended in PBS containing 0.5 mM Mg⁺⁺, 1 mM Ca⁺⁺, 5 mM glucose and 0.1% human serum albumin (PBSG-HSA).

Enumeration of S. epidermidis in Fibrin Gels

200 μl PBS (no Ca++ and Mg++) containing 5 mg/ml trypsin, with or without 20 mM EDTA and 20 μM cytochalasin D, pH 10.4, 4° C., was added to each gel for 10 min to allow diffusion of phagocytosis inhibitors into the gel. The gels were then incubated at 37° C. for 18 min. The liquefied gels were diluted with sterile distilled water, and incubated for another 5 min at 37° C., as described 84, to completely lyse the neutrophils. Serially diluted samples were plated on TSB agar plates, incubated overnight at 37° C., and colonies were counted manually.

Transmission Electron Microscopy

To facilitate processing for microscopy we used 50% autologous plasma to form gels (40 μl in volume). To increase the frequency of neutrophils and S. epidermidis interactions the gels contained 4μ 10⁸ neutrophils/ml (pre-incubated in medium with or without 20 μM cytochalasin D for 15 min at room temperature), 2μ 10⁸ CFU/ml S. epidermidis, and 20 μM cytochalasin D, where indicated. The gels were incubated for 60 min at 37° C., fixed with 2.5% glutaraldehyde at 4° C., and then with 1% OsO₄, stained en block with 1% uranyl acetate, dehydrated, and embedded in Epon. Sections ˜600 nm thick were cut, stained sequentially with lead citrate and uranyl acetate, and examined in a Phillips 1200 transmission electron microscope.

Data Analysis

Bacterial killing (%)=(1-[S. epidermidis](90min,+neutrophils)/[S. epidermidis] (90 min, no neutrophils])×100%

Statistics

Experiments were performed at least three times in duplicate and are reported as the means±SEM for the number of experiments indicated. Significance was obtained using two-sample paired Student's t test.

Quantitative recovery of S. epidermidis from Fibrin Gels

Rotstein, et al., reported that neutrophils killed 90% of E. coli embedded in gels formed with 1 mg/ml fibrinogen. Rotstein, O. D., Pruett, T. L. & Simmons, R. L. Fibrin in peritonitis. V. Fibrin inhibits phagocytic killing of Escherichia coli by human polymorphonuclear leukocytes. Ann Surg 203, 413-9 (1986). In their study, bacteria were recovered from these gels after trypsin digestion. However, they did not report the efficiency of recovery of bacteria from these gels, or test the effects of digestion of the gels on recovery of viable bacteria. Therefore, the recovery of S. epidermidis from fibrin gels containing these bacteria, normal human serum and the indicated number of neutrophils was examined (Table 1). The gels were digested with 5 mg/ml trypsin in PD (PBS without Ca⁺⁺ or Mg⁺⁺) containing cytochalasin D and EDTA to block phagocytosis of bacteria during trypsin digestion of the gels. EDTA was used to block interaction between C3-coated bacteria and neutrophil integrins. After lysis of the gels, the resulting suspensions were diluted and plated on nutrient agar and the number of bacteria counted after 18 h incubation at 37° C.

Over 99% of S. epidermidis were recovered from gels containing S. epidermidis alone or S. epidermidis and 4×10⁶/ml neutrophils, whether or not EDTA, and cytochalasin D, were included in the lysis buffer (Table 1). Wright, S. D. & Silverstein, S. C. Tumor-promoting phorbol esters stimulate C3b and C3b′ receptor-mediated phagocytosis in cultured human monocytes. J. Exp Med 156, 1149-64. (1982); Barkalow, K. & Hartwig, J. H. The role of actin filament barbed-end exposure in cytoskeletal dynamics and cell motility. Biochem Soc Trans 23, 451-6. (1995). However, when using a ten-fold greater number of neutrophils (4×10⁷/ml), only 75% of S. epidermidis were recovered from gels lysed in the absence of EDTA and cytochalasin D, a significant decrease in recovery (p <0.01) as compared to >99% recovery from gels lysed in the presence of these inhibitors (Table 1). Further studies showed >99% recovery of S. epidermidis when cytochalasin D was the sole inhibitor in the lysis buffer (not shown). Thus, inclusion of cytochalasin D in the lysis buffer ensures full recovery of S. epidermidis from fibrin gels, even when the gels contained neutrophils at a concentration 8-fold in excess of that used in subsequent experiments.

TABLE 1
Effect of EDTA & cytochalasin D on recovery of S. epidermidis
from fibrin gels containing 4 × 10⁶ or 4 × 10⁷neutrophils/ml of gel

| | |
|---|---|
| [Neutrophil] | S. epidermidis recovered (%) |

| per ml fibrin gel | Cyto D + EDTA | no inhibitors |
|---|---|---|
| 0 | 98 ± 1 | 97 ± 2 |
| 4 × 10⁶ | 98 ± 2 | 102 ± 2 |
| 4 × 10⁷ | 100 ± 3 | 74 ± 3** |

Fibrin gels (1500 μm thick, 100 μl in volume) containing S. epidermidis (1×10⁵ CFU/ml), 40% normal human serum, and the indicated concentrations of neutrophils were incubated with 200 μl PD containing trypsin (5 mg/ml) alone (no inhibitors), or with PD containing trypsin (5 mg/ml), EDTA (20 mM) and cytochalasin D (20 μM) (Cyto D+EDTA). Shown is the percent of the inoculum (1×10⁵CFU/ml) recovered from digested gels. Data represent the means±SEM of three experiments, each performed in duplicate. **p <0.01 compared to control of zero neutrophil.

Derivation of Equations

Rate Constants of Neutrophil Bacterial Killing

It is assumed that neutrophils kill bacteria in a second-order collisional process, in which the neutrophils are not consumed; i.e.,

$$B + P {\to }^{k} {B}^{*} + P \qquad ( 3 \text{-} 1 )$$

where k is a second-order rate constant, B* is a bacterium, B* is a killed bacterium, and P is a neutrophil. At the same time, the bacteria are replicating in a first-order reaction characterized by the first-order rate constant, g; i.e.,

$$B {\to }^{g} 2 B \qquad ( 3 \text{-} 2 )$$

The change in the concentration of viable bacterial (b) with time (t) is
db/dt=−kpb+gb (3-3)
where p (neutrophil concentration ) is assumed not to change.
bₜ=b₀e^{-kpt+gt} (3-4)
is obtained where b₁ is the concentration of viable bacteria after incubation time t, and b₀ is the initial concentration of viable bacteria.

Equation 3-4 can also be expressed with t factored out:
bₜ=b₀e^{k′t} (3-5)
where
k′=−k p+g. (3-6)
k=(−k′+g)/p (3-6-1)

The Critical Neutrophil Concentration (CNC)

Equation 3-3 describes the rate at which bacterial concentration change as the sum of the rates of bacterial killing and bacterial growth. When the rate of bacterial killing is equal to the rate of bacterial growth, then
db/dt=−kpt+gt=0.
or
kpt=gt,
and
p=g/k.

This p (equal to g/k) was termed the critical neutrophil concentration (CNC). Thus, the CNC is the neutrophil concentration at which the rate of bacterial killing is equal to the rate of bacterial growth.

### Example

Confocal Microscopy Shows Partial C3 Opsonization of 10-day-old Biofilms

In vivo, biofilms persist for days to weeks. Therefore, the relationship between biofilm age and efficiency of opsonization of bacteria in the biofilm was tested. To measure C3 opsonization of bacteria in 10-day-old biofilms, a mucoid forming strain of S. epidermidis was grown for 10-day under shear stress, a condition that mimics biofilm formation on venous catheters. Whole pieces of 10-day-old biofilms were then incubated with normal serum at 37° C. for 30 min, examined for C3 staining by immunofluorescence confocal microscopy. Bacteria were revealed by Syto-13, a nucleic acid binding fluorescent dye that stains each bacterium in the biofilms.

The 10-day-old S. epidermidis biofilms were large, and contained aggregates of bacteria that could not be dispersed by vigorous vortexing, one of the characteristics that distinguishes mature from immature biofilms and from typical bacterial colonies. As shown in FIG. 1, S. epidermidis in 10-day-old biofilms (stained green with Syto-13) appeared separated with channels (dark spaces) in between individual bacterium, another characteristics typical of biofilm structure. C3 (red) appeared deposited only on part of the biofilms. C3 deposited on the surface of biofilm unevenly and varied with from place to place along the biofilm's surface (FIG. 1A). It also stained bacteria in the outer portion of the biofilm, but was absent from about ⅔ of the cross-section through the middle of the biofilm (FIG. 1B). C3 deposition directly on the cell wall of a bacterium is indicated by a red ring of anti-C3 fluorescence around the bacterium. Green fluorescence was absent from the center of some red rings, suggesting the presence of the wall of a lysed bacterium. Most of the red staining was diffuse, especially on the surfaces of the biofilms, indicating complement deposition on the biofilm's extracellular matrix (i.e., exopolysaccharides).

Ultrastructural Appearance of the Biofilms

In vivo, neutrophils often accumulate in large numbers near sites of biofilm infection without actually penetrating into the layer containing the biofilm or into the biofilms themselves (Daniel Lew, University of Geneva, personal communication). It has been reported that biofllm exopolysaccharides inhibit neutrophil chemotactic activity. However, it also is possible that insufficient C5a is released from biofilms to attract neutrophils to them. The fibrin gel system provided an opportunity to examine this idea. Fibrin gels were prepared (40 μl in volume and 600 μm in thickness) containing 10-day-old S. epidermidis biofilms and 40% normal serum, placed 1.6×10⁶ neutrophils on top of these gels (a final concentration of 40×10⁶/ml with all the neutrophils penetrated into the gels), incubated for 6 h at 37° C., and the neutrophil penetration and contact with biofilms by transmission electron microscopy was examined.

Examination of thin sections of fibrin gels fixed immediately after preparation (0 h) showed they contained biofilms with viable bacteria that were septated and had well demarcated nucleoids (FIG. 2C). In contrast, thin sections of gels fixed after 6 h incubation showed that numerous neutrophils had polarized, a shape that indicates neutrophil activation, and stacked up one after another on the biofilm's surface. Each of these biofilm-adherent neutrophils exhibited an elongated pseudopod in close contact with ˜2 μM of the biofilm's surface. The cytoplasm of these neutrophils was devoid of granules and most other cyto-membranes, and, in contrast to neutrophils not in contact with the biofilm, contained few if any phagocytosed bacteria (Compare FIG. 2A). The bacteria in the portions of the biofilm underlying zones of neutrophil adhesion showed electron lucent holes, suggesting absence of DNA-containing nucleoids. Moreover, few of these bacteria exhibited the septa characteristic of dividing S. epidermidis. In contrast, as noted above, most bacteria in biofilms harvested at time 0 were septated and contained a fibrillar nucleoid in their cytoplasm (Compare FIGS. 2A, B and C). These differences suggested that by 6 h, the neutrophils and/or their secretory products were adversely affecting S. epidermidis.

Quantitative Analysis of Complement Activation by 1-, 5-, and 10-day-old Biofilms

Meluleni, et al., reported that one-day-old P. aeriginosa biofilms stimulate complement activation and deposition of C3 on the biofilm. Meluleni, G. J., Grout, M., Evans, D. J. & Pier, G. B. Mucoid Pseudomonas aeruginosa growing in a biofilm in vitro are killed by opsonic antibodies to the mucoid exopolysaccharide capsule but not by antibodies produced during chronic lung infection in cystic fibrosis patients. J. Immunol 155, 2029-38. (1995). However, they did not examine whether the age of the biofilm affected IgG and/or C3 deposition. To assess these parameters, 1-, 5-, and 10-day-old biofilms were incubated in 50% normal human serum for 30 min at 37° C., washed, sonicated to create a suspension of planktonic bacteria, and incubated with FITC-labeled anti-human C3 monoclonal antibody and/or PE-labeled anti-human IgG. The bacteria then were washed and examined by flow cytometry to determine both the fraction of opsonized bacteria and the relative amounts of C3 and IgG bound to them.

While essentially all S. epidermidis in 1-day-old biofilms bound FITC-labeled anti-human C3 monoclonal antibody, only ˜50% of S. epidermidis in 5-day and 10-day-old biofilms did so (FIG. 3). As a control, planktonic S. epidermidis isolated from sonicated un-opsonized biofilms were incubated at the same bacterial concentration for the same time with the same serum concentration as the biofilms. Virtually all planktonic S. epidermidis from 1, 5, and 10-day-old biofilms bound FITC-labeled anti-human C3 monoclonal antibody.

S. epidermidis is a commensal bacterium that is part of the normal skin flora. Therefore, all humans are exposed to S. epidermidis antigens and almost all sera from normal humans contain anti-S. epidermidis IgG. Nearly 100% of S. epidermidis from 1- and 5-day old biofilms incubated in normal human serum, sonicated and then incubated with PE-labeled anti-IgG stained for bound human IgG. S. epidermidis from 10-day-old biofilms bound significantly less IgG than planktonic controls.

There were no differences in the percentages of S. epidermidis from 1-day-old biofilms incubated in normal human serum that stained with FITC-labeled anti-human C3 monoclonal antibody and PE-labeled anti-human IgG than of planktonic bacteria released from these biofilms and then incubated in normal human serum. Similarly, there were no differences in the amounts of C3 and IgG deposited on the surfaces of S. epidermidis from 1-day-old biofilms incubated in normal human serum vs. S. epidermidis that were released by sonication from 1-day-old biofilms and then incubated in normal human serum.

Two important conclusions can be derived from these experiments. First, they show the amounts of C3 and IgG deposited on biofilm bacteria are inversely proportional to the age of the biofilm. S. epidermidis in 5-day-old biofilms bound less C3 than S. epidermidis in 1-day-old biofilms, S. epidermidis in 10-day old biofilms bound less C3 than S. epidermidis in 5- or 1-day-old biofilms; and less IgG than S. epidermidis in 1-day-old biofilms. Second, studies of the interactions of serum opsonins, and probably of neutrophils, with 1-day-old biofilms do not provide an accurate picture of their interactions with more mature biofilms.

Neutrophil Killing of S. epidermidis from 10-day-old Bioflims Incubated with Normal Serum

To determine whether the reduced C3 binding to S. epidermidis affects the efficiency with which these bacteria are killed by neutrophils, 10-day-old S. epidermidis biofilms were incubated in PBS-GHSA containing 50% normal human serum for 30 min at 37° C., washed to remove serum, bacteria from the biofilms was released by sonication. Killing of these bacteria by human neutrophils in stirred suspensions with or without the addition of normal serum was then compared. Sixty percent S. epidermidis were killed in the absence of added serum, whereas more than 90% S. epidermidis were killed with added serum. Since both the percentage of bacteria coated with C3 and IgG and the amounts of C3 and IgG bound to these bacteria were reduced in 10-day biofilms, further work is required to determine whether the reduction in one or the other opsonins is of paramount importance.

Neutrophil Killing of S. epidermidis in 5-day-old Biofilms in Fibrin Gels

To measure the efficiency with which neutrophils kill bacteria that are embedded in biofilms, new experimental methods were developed. It is important to determine the initial number of viable bacteria in biofilms (b₀), as it is necessary for measuring k (Eq. 3-4). However, the principal problem to be overcome was that b₀ in each piece of biofilms depends on the size of the biofilm, and conventional microbiological plating method for assaying b₀ would require disruption of biofilms, making it impossible to measure killing of bacteria embedded in whole pieces of biofilms. In preliminary experiments, it was discovered that the initial viable number of bacteria can be determined without disruption of biofilms by fluorescence of BCECF-labeled biofilms. Incubation of S. epidermidis biofilms in BCECF-AM-containing buffer resulted in trapping of BCECF in the biofilm bacteria. The fluorescence of these BCECF-AM-labeled biofilms correlated linearly with their content of viable bacteria (FIG. 5). By use of standard curves relating fluorescence of bacteria in a biofilm to its content of viable bacteria, the number of bacteria initially present in a single piece of biofilm was determined. This method was used for the studies described below.

To determine the efficiency of neutrophil killing of S. epidermidis in biofilms (k), fibrin gels were formed containing pieces of BCECF-labeled 5-day-old S. epidermidis biofilms, 13×10⁶ or 26×10⁶ neutrophils/ml and 40% normal serum, and incubated these gels for 3 h at 37° C. The number of viable S. epidermidis remaining in the gels was measured, as described. In the absence of neutrophils, the number of S. epidermidis in 5-day-old biofilms increased 3-fold during the 3 h. In the presence of 13×10⁶ and 26×10⁶/ml neutrophils, 92% and 98% of the bacteria, respectively, were killed (FIG. 5). The k value calculated from these experiments was 1×10⁻⁹ ml/neutrophilmin. Further work is needed to determine whether neutrophils can kill S. epidermidis in 10-day-old biofilms, and to assess whether they must penetrate the biofilm to do so.

New methods for quantitative analysis of the efficiency and extent of C3 and IgG opsonization of S. epidermidis in biofilms, and for comparing the efficiency of killing of biofilm vs. planktonic bacteria by human neutrophils have been described above. These experiments show that the age of S. epidermidis biofilms is an important determinant of complement and IgG opsonization of S. epidermidis in them. All S. epidermidis in 1-day-old biofilms become coated with C3. However, only 50% of these bacteria in 5- and 10-day-old biofilms become opsonized with C3. They bind significantly (25% and 50%) smaller amounts of C3 than their planktonic counterparts from the same biofilms. Together with the finding that >90% of 10-day-old biofilm S. epidermidis can be killed by neutrophils once they have been released from the biofilms, these findings suggest that growth of S. epidermidis in a biofilm does not affect the ability of IgG and C3 to bind to it, and does not change the resistance of these bacteria to killing by neutrophils. These are important conclusions. They suggest that if drugs or pathways can be identified to lyse highly mature biofilms, the released planktonic bacteria will be opsonized and killed by neutrophils. Meluleni, et al., came to a similar conclusion with respect to neutrophil killing of P. aeruginosa in 1-day-old biofilms. Meluleni, G. J., Grout, M., Evans, D. J. & Pier, G. B. Mucoid Pseudomonas aeruginosa growing in a biofilm in vitro are killed by opsonic antibodies to the mucoid exopolysaccharide capsule but not by antibodies produced during chronic lung infection in cystic fibrosis patients. J Immunol 155, 2029-38. (1995).

Further work is needed to determine the reasons for reduced C3 deposition on S. epidermidis in 5- and 10-day-old biofilms. It seems unlikely that it is due to the inability of C3 to penetrate into the interior of the biofilm since IgG, a protein only slightly smaller than C3, (IgG=150 kd vs. C3=185 kd) penetrates 5- and 10-day-old biofilms fairly efficiently (FIGS. 6-1, 3, & 4). It seems more likely that proteases or other components of the biofilm matrix degrade C3, or inhibit its activation.

At the time of fibrin gel formation, neutrophils are randomly and isotropically distributed throughout the gel. The finding that C3 becomes fixed to S. epidermidis biofilms and that neutrophils collect in large numbers around them (FIG. 2), is strong presumptive evidence that chemoattractants (e.g., C3a and C5a), stimulate neutrophils to migrate toward the biofilms. Whether these chemoattractants are sufficient to stimulate neutrophils to adhere to the biofilms, or whether they adhere only to portions of the biofilm coated with opsonic ligands (e.g., C3b, C4b, IgG), now can be resolved using the fibrin gel system, sera selectively depleted of anti-S. epidermidis IgG and/or of one or more complement components, and immunocytochemical methods.

The observation that neutrophils become very closely apposed to the surfaces of biofilms, display a polarized phenotype, and degranulate completely (FIG. 2), raises the possibility that they form “protected compartments” on the biofilms and secrete bacteriostatic and bactericidal substances (e.g., lactoferrin, defensins, elastase, myeloperoxidase and H2O2), into them. Wright, S. D. & Silverstein, S. C. Phagocytosing macrophages exclude proteins from the zones of contact with opsonized targets. Nature 309, 359-61. (1984). By this means neutrophils may be able to damage and kill bacteria otherwise protected from engulfment by the biofilm's exopolysaccharide matrix.

Measurement of the bactericidal efficiency of neutrophils requires one to know the initial concentration of bacteria. Prior to the studies reported here, there were no methods for determining the number of bacteria in a biofilm without destroying it. Indeed, the only previous study of neutrophil bactericidal activity against bacteria (i.e., P. aeruginosa) in a biofilm relied on an estimate of the average number of CFU of P. aeruginosa in biofilms of roughly comparable size. Meluleni, G. J., Grout, M., Evans, D. J. & Pier, G. B. Mucoid Pseudomonas aeruginosa growing in a biofilm in vitro are killed by opsonic antibodies to the mucoid exopolysaccharide capsule but not by antibodies produced during chronic lung infection in cystic fibrosis patients. J. Immunol 155, 2029-38. (1995). Use of BCECF-AM, allowed an accurate measure of the number of CFU of S. epidermidis in S. epidermidis biofilms without disrupting the biofilms (FIG. 5). Using this method, it was shown that the rate constant for neutrophil killing of S. epidermidis in 5-day-old biofilms embedded in fibrin gels was ten to twenty times smaller (i.e., 1×10⁻⁹ ml /neutrophil/min), than for killing a similar number of planktonic bacteria under the same experimental conditions (i.e., 1-2×10⁻⁸ ml/neutrophil/min). In these killing studies, neutrophils at concentrations over 10×10⁶/ml were used. At neutrophil concentrations >10⁷/ml, neutrophil killing of planktonic bacteria is relatively insensitive to the presence or absence of C5a. With planktonic bacteria, the k values obtained at these neutrophil concentrations primarily reflect the efficiency of phagocytosis and intracellular killing. Absent information about the mechanism(s) of neutrophil killing of biofilm bacteria, it is not possible to say which steps in the killing process affect the value of k. Nonetheless, the finding that k for neutrophil killing of S. epidermidis in biofilms is smaller than for neutrophil killing of planktonic S. epidermidis provides the first quantitative measure of the effect of biofilms on neutrophil bactericidal activity.

The experiments described here provide the first quantitative estimates of the extent of C3 and IgG opsonization of bacteria in a biofilm, the first evidence that the extent and efficiency of opsonization of bacteria in a biofilm are related to the biofilm's age, the first demonstration that neutrophils can kill bacteria in relatively mature (e.g., 5-day-old) biofilms, the first indication that neutrophils can be stimulated to adhere in large numbers to 10-day-old biofilms; and the first suggestion that they may be able to kill bacteria in a biofilm without phagocytosing them.

Critical Neutrophil Concentration

Materials and Methods

S. epidermidis

S. epidermidis H753 was obtained, cultured, and assayed as described in Li, Y., et al., The bacterial peptide N-formyl-Met-Leu-Phe inhibits killing of Staphylococccus epidermidis by human neutrophils in fibrin gels. J. Immunol. 168, 816-24 (2002).

Normal Human Plasma-derived Serum (NS)

NS was prepared from AB plasma (New York Blood Center, New York, N.Y.) as described and the serum contained anti-So epidermidis IgG and complement. Li, Y., et al. The bacterial peptide N-formyl-Met-Leu-Phe inhibits killing of Staphylococccus epidermidis by human neutrophils in fibrin gels. J. Immunol. 168, 816-24 (2002).

Neutrophil Killing of S. epidermidis in Fibrin Gels

Fibrin gels (100 μl in volume) containing 1 mg/ml purified human fibrinogen (American Diagnostica Inc, Greenwich, Conn.), human neutrophils, S. epidermidis, and NS (40% v/v, a concentration optimal for neutrophil bactericidal activity in fibrin gels [data not shown]), were prepared and incubated for 90 min at 37° C. to measure neutrophil bactericidal activity as described in Li, Y., et al. The bacterial peptide N-formyl-Met-Leu-Phe inhibits killing of Staphylococccus epidermidis by human neutrophils in fibrin gels. J. Immunol. 168, 816-24 (2002). Control experiments showed that >99% of viable S. epidermidis. were recovered from fibrin gels, even in the presence of >10⁸ neutrophils, and that >98% of neutrophils were viable (determined by exclusion of propidium iodide [Molecular Probes, Eugene, Oreg.]) after 90 min incubation in fibrin gels containing 106 01 108 CFU/ml S. epidermidis. Li, Y., et al. The bacterial peptide N-formyl-Met-Leu-Phe inhibits killing of Staphylococccus epidermidis by human neutrophils in fibrin gels. J. Immunol. 168, 816-24 (2002). The fibrinogen concentration in lymph draining normal human or rabbit skin is −30% of that in plasma, and sufficient to form a clot. Le, D.T., et al. Hemostatic factors in rabbit limb lymph: relationship to mechanisms regulating extravascular coagulation. Am. J. Physiol. 274, H769-76 (1998); Olszewski, W. L. and Engeset, A. Haemolytic complement in peripheral lymph of normal men. Clin. Exp. Immunol. 32, 392-8 (1978). Normal plasma contains¹⁸ 3 mg/ml. fibrinogen. Thus, the fibrinogen concentration used to form these gels (1 mg/ml) is close to that found in in vivo (i.e., 3 mg/ml×30%=0.9; mg/ml). Similarly, the concentrations of C3, C5 and IgG in lymph are between 10 and 25% of those in plasma, and sufficient to support nearly optimal neutrophil killing of S. epidermidis in fibrin gels. Olszewski, W. L. and Engeset, A. Haemolytic complement in peripheral lymph of normal men. Clin. Exp. Immunol. 32, 392-8 (1978); Elsbach, P., et al. Inflamation: Basic Principles and Clinical Correlates (eds. Gallin, J. I., Snyderman, R. and Nathan, C.) 801-817 (lippincott-Raven, Philadelphia, Pa. 1999).

Intercellular distances Fibrin gels were formed by placing 10 μl buffer containing 1 mg/ml fibrinogen, 10% NS, 1 U/ml thrombin (Sigma, St. Louis, Mo.), 6 μM Syto-13 (Molecular Probes, Eugene, Oreg.), and neutrophils on a 12-well multi-spot microscope slide (Shandon Inc. Pittsburgh, Pa.). The gels thus formed were ˜60-80 ˜m thick. Z-series images of 20 ˜m-thick optical sections (optimal for resolving the relative locations of adjacent neutrophils in all directions) were captured at 20 ˜m interval by confocal fluorescence microscopy using a 25×oil-immersion objective. Intercellular distances between a randomly chosen neutrophils and five to six nearest cells in the same or adjacent optical section were determined using LSM 5 Image Brower (Carl Zeiss, USA). The mean and SEM of six such determinations were calculated.

Equations

bt=b0^{e-kpt+gt}(Eq. 1) Li, Y., et al. A critical concentration of neutrophils is required for effective bacterial killing in suspension. Proc. Nat'l. Acad. Sci. U.S.A. 99, 8289-94 (2002).
k′=(−kp+g) (Eq. 2)
bₜ₊₆₀ₘᵢₙ=bₜe^{k′60min} (Eq. 3)
k′=Ln (bₜ₊₆₀ₘᵢₙ/_{bt})/60 min (Eq. 4)
CNC=g/k (Eq. 5)

Calculation of k, g and CNC for E. coli-infected Rabbit Dermis

Movat, et al., 6 7 reported that virtually all neutrophils that migrated into E. coli-infected dermis of rabbits were contained in the 0.2-cm thick segment of dermis in a 1.5-cm 5 diameter full thickness biopsy of rabbit skin. The volume of dermis in each E. coli-inoculated skin site was therefore 0.353 cm3 or 0.353 ml, and the E. coli concentration (bt,) at each site was 1/0.353 ml×E. coli number per skin site6 (Table 2). Similarly, the number of neutrophils that migrated each hour into E. coli-infected dermis of normal rabbits6 were converted to neutrophil concentrations accumulated per hour (number 0 f neutrophils/0.353 ml), and the concentrations accumulated per hour were then summed to give the cumulative neutrophil concentration (pt)(Table 2 and FIG. 3b). Since Pt varied, the average Pt. t+60 min was calculated and used for the calculation of k. g of 0.017 (min-1) was obtained by solving Eq. 1 with p=0 and the concentrations of E. coli recovered from dermis of rabbits rendered neutropenic <<5×105 neutrophils/ml blood) by cyclophosphamide treatment: bo=5.7×107 CFU/ml dermis, and b₆₀ₘᵢn=1.2×108 CFU/ml dermis. k was determined by solving Eq. 2-4 using bl, bₜ₊₆₀ₘᵢₙ, Pt. t+60min, and g=0.017 (min⁻¹). CNC was calculated using Eq. 5.

Neutropliil Extraction Efficiency

Neutrophil extraction efficiency (NEE) was calculated by dividing the concentration of neutrophils accumulated in 1 ml of E. coli inoculated rabbit dermis each hr after infection (P_{t+60min-Pt}) by the total number of neutrophils delivered in the same hour to 1 ml E. coli-inoculated rabbit dermis (FIG. 3b). Total number of neutrophils delivered=basal blood flow of 3.6 ml/g/hr in uninfected rabbit skin 8×the fold increase in blood flow in E. coli-infected rabbit dermis (FIG. 3c)×blood neutrophil concentration at various times after E. coli inoculation. Cybulsky, M. I., Cybulsky, I. J. & Movat, H. Z. Neutropenic responses to intrademal injections of Escherichia coli. Effects on the kinetics of polymorphonuclear leukocyte emigration. Am J Pathol 124, 1-9. (1986); Kopaniak, M. M. & Movat, H. Z. Kinetics of acute inflammation induced by Escherichia coli in rabbits. II. The effect of hyperimmunization, complement depletion, and depletion of leukocytes. Am J Pathol 110, 13-29. (1983). Blood neutrophil concentration in uninfected rabbits=2.5×106/ml[ref.7]).

Results

Neutrophil Concentration Determines Their Efficiency in Killing S. epidermidis in Fibrin Gels

Neutrophils and S. epidermidis were co-embedded at the concentrations indicated (FIG. 1a) in fibrin gels containing normal human serum. The gels were incubated for 90-min at 37° C., lysed, and their content of viable S. epidermidis assayed. At neutrophil concentrations ranging from 10⁵ to 10⁷ /ml fibrin gel, the number of bacteria remaining viable at 90 min compared to the initial bacterial inoculum depended primarily on the initial concentration of neutrophils in these gels (FIG. 1). At 4×10⁶ neutrophils/ml, fewer viable bacteria were recovered after 90 min than were present in the inoculum, even when there were 108 CFU S. epidermidis/ml gel, and the ratio of neutrophils:bacteria was 1:25 (FIG. 1a). Conversely, at 4×10⁵ neutrophils/ml, more viable bacteria were recovered after 90 min than were present in the inoculum, even when there were only 103 CFU S. epidermidis/ml gel, and the ratio of neutrophils:bacteria was 400:1 (FIG. 1a). Control experiments showed that >99% of bacteria embedded in fibrin gels with or without neutrophils were recovered from these gels at zero time. Le, D. T., Borgs, P., Toneff, T. W., Witte, M. H. & Rapaport, S. I. Hemostatic factors in rabbit limb lymph: relationship to mechanisms regulating extravascular coagulation. Am J Physiol 274, H769-76 (1998).

FIG. 1a reports the difference between the number of viable S. epidermidis remaining after incubation with neutrophils (b₉₀ₘᵢₙ, and the number of bacteria in the inoculum (bₒ)(i.e., b₉₀ₘᵢₙ/b₀). This difference does not reflect the total number of bacteria killed, since even when the neutrophil concentration was insufficient to block net bacterial growth, some bacteria were killed. To obtain a more complete picture of the relationships between neutrophil and bacterial concentration and bacterial killing, we calculated total bacterial killing at neutrophil concentrations ranging from 10⁵ to 10⁷ ml, and bacterial concentrations ranging from 103 to 108 CFU/ml. For bacterial inocula of 10³ to 10⁶ CFU/ml, the fraction of S. epidermidis killed ranged from −25% at 4×105 neutrophils/ml, to >99% at 107 neutrophils/ml (FIG. 1b). Neutrophil bactericidal efficiency declined with bacterial inocula >106 CFU/ml. Nonetheless, even at 10⁸ CFU S. epidermidis/ml, neutrophils at concentrations as low as 4×10⁵ /ml killed a small fraction (−10%) of S. epidermidis.

S. epidermidis killing increased with neutrophil concentration at all bacterial concentrations (FIG. 1b). This increase was related to the absolute neutrophil concentration rather than the ratio of neutrophils to bacteria. For example, 4×10⁶ neutrophils/ml fibrin gel killed >90% of inocula containing 10³ to 10⁷ CFU S. epidermidis/ml fibrin gel (ratios of neutrophils:bacteria of 4000:1 and 1:2.5, respectively), while 4×10⁵ neutrophils/ml killed only −20-25% of inocula containing 10³ to 10⁷ CFU S. epidermidis/ml (ratios of neutrophils:bacteria of 400:1 and 1:25, respectively)(FIG. 1b). These results confirm that the efficiency of neutrophil bactericidal activity in three-dimensional matrices is highly dependent on the neutrophil concentration.

Determination of k, the rate constant for neutrophil killing of bacteria in fibrin gels. Eq. 1 assumes a random distribution of a constant number of viable neutrophils throughout the course of an experiment. As described in Methods and in FIG. 2 legend, we confirmed experimentally that neutrophils were distributed uniformly in fibrin gels (FIG. 2), and were viable throughout the 90 min course of experiments (not shown).

Eq. 1 states that the log of the concentration of viable bacteria remaining after incubation with neutrophils (br) is a linear function of neutrophil concentration. Plots of the log of b, in fibrin gels after a 90 min incubation vs. neutrophil concentration appeared to be linear for all neutrophil and bacterial concentrations tested (FIG. 1c, symbols). Non-linear regression analyses of these data with Eq. 1 yielded closely fitted functions for the experimentally determined results (FIG. 1c, solid lines). The slope of each curve yields k×t. k was 10×10⁻⁹ ml/neutrophil/min for S. epidermidis inocula ˜10⁶ CFU/ml, and 7×10⁻⁹ and 2×10⁻⁹ ml/neutrophil/min for S. epidermidis inocula of 10⁷ and 10⁸ CFU/ml, respectively (Table 1).

Fitting the linear function k=−q×bₒ+kₒ to values of k obtained at S. epidermidis inocula of 10³ to 10⁸ CFU/ml yielded a line that closely fits the data with a slope (q) of 8×10⁻¹⁷ (R2=1), indicating that S. epidermidis concentration has an extremely small effect on k. The effect was so small that for S. epidermidis inocula ≧10⁶ CFU/ml, k was constant (Table 1). For inocula >10⁶ CFU/ml, a 100-fold increase in inoculum (from 10⁶ to 10⁸ CFU/ml), resulted in only a 5-fold decrease in k (from 10×10⁻⁹ to 2×10⁻⁹ ml/neutrophil/min, Table 1).

Determination of the CNC for Killing of S. epidermidis in Fibrin Gels

The CNC is given by g/k. The CNC required to block growth of S. epidermidis inocula of 103 -106 CFU/ml fibrin gel was 106 neutrophils/ml (Table 1), and 2×106 and 4×106 neutrophils/ml gel for S. epidermidis inocula of 107 and 108 CFU/ml gel, respectively.

The finding that both k and CNC changed at bacterial concentrations >106 CFU/ml fibrin gel, and >107 CFU/ml in stirred suspensionsl, appears to contradict the assertion that killing efficiency is strictly dependent on neutrophil concentration. However, Eq. 1 accurately describes neutrophil bactericidal activity at all bacterial concentrations tested (FIG. 1c). Since both g (Table 1), andp were constant (neutrophil viability remained >98% throughout the course of experiments), the increase in CNC at bacterial concentrations >106 CFU/ml was solely due to a decrease in k. The reason(s) for this decrease is unknown.

Phagocytosis is Required for Killing of S. epidermidis in Fibrin Gels

In stirred suspensions, neutrophils must phagocytose bacteria to kill them. Li, Y., Karlin, A., Loike, J. D. & Silverstein, S. C. A critical concentration of neutrophils is required for effective bacterial killing in suspension. Proc Natl Acad Sci USA 99, 8289-94. (2002). Two lines of evidence indicate that phagocytosis, not neutrophil secretory products, mediates killing of S. epidermidis in fibrin gels. First, there was no decrease in CNC as the neutrophil concentration increased from 10⁶ to 10⁷/ml (Table 1). This is inconsistent with a significant role for neutrophil secretory products in bacterial killing. Second, cytochalasin D, which facilitates neutrophil secretions, blocked both phagocytosis (as measured by electron microscopy), and killing of S. epidermidis in fibrin gels at all bacterial (10⁵ to 2×10⁸ CFU/ml) and neutrophil concentrations (10⁶ to 4×10⁸/ml) tested (data not shown). Gallin, J. I. & Snydennan, R. (eds.) Inflammation: basic principles and clinical correlates (Lippincott Williams & Wilkins, Philadelphia, 1999).

The Values of k and CNC for E. coli in Rabbit Dermis in vivo Are Similar to those for S. epidermidis in Fibrin Gels in vitro

Movat, et al., inoculated rabbits intra-dermally with live E. coli and monitored blood neutrophil concentration and CFU of E. coli in these dermal sites 0-8 hr thereafter. Movat, H. Z., Cybulsky, M. I., Colditz, I. G., Chan, M. K. & Dinarello, C. A. Acute inflammation in gram-negative infection: endotoxin, interleukin 1, tumor necrosis factor, and neutrophils. Fed Proc 46, 97-104. (1987). To compare Movat, et al.'s, findings with those reported in FIG. 1 for fibrin gels, we converted Movat, et al.'s, data to concentrations of neutrophils and E. coli per ml dermis (FIGS. 3a & b). We solved for k using Eq. 1, and used the values of k and g to calculate the CNC required to block growth of E. coli in rabbit dermis in vivo.

Movat, et al., reported that neutrophils began migrating into the dermis of normal rabbits −30 min after inoculation of 2×107 CFU live E. coli. We calculate that the neutrophil concentration was 2.3×10⁶ and 12×10⁶/ml dermis, 1 and 2 hr post E. coli inoculation, respectively, and that it continued to increase at an ever decreasing rate for 6 hr more (FIG. 3b). The E. coli concentration increased from 5×10⁷ CFU/ml dermis initially to 1.1×10⁸ CFU/ml dermis at one hr, was also −1.1×10⁸ CFU/ml dermis at the end of two hr, and then decreased to 5×10⁶ CFU/ml dermis over the ensuing 6 hr (FIG. 3a).

In contrast, in dermis of neutropenic rabbits (−5×105 neutrophils/ml blood [Cybulsky, M. I., Cybulsky, I. J. & Movat, H. Z. Neutropenic responses to intradermal injections of Escherichia coli. Effects on the kinetics of polymorphonuclear leukocyte emigration. Am J Pathol 124, 1-9. {1986).]), E. coli grew to a concentration of 2×108 CFU/ml dermis at 1 hr, and increased continuously over the ensuing 7 hr, albeit at a slower rate (FIG. 3a). Kopaniak, M. M. & Movat, H. Z. Kinetics of acute inflammation induced by Escherichia coli in rabbits. II. The effect of hyperimmunization, complement depletion, and depletion of leukocytes. Am J Pathol 110, 13-29. (1983), reported that almost no neutrophils immigrated into the dermis of neutropenic rabbits in the first hr after E. coli inoculation. Therefore, we used E. coli growth in the first hr to calculate g in rabbit dermis. g was 0.017/min, equivalent to an E. coli doubling time of 40 min.

Substituting the dermal concentrations of neutrophils (P) and E. coli at the time of inoculation (bₒ), and at various times thereafter (br), and of g into Eq. 1 (see Methods), we determined a value of k of 2.2-2.3×10⁻9 ml/neutrophil/min for neutrophil killing of −108 CFU/ml E. coli in rabbit dermis (Table 2). This is very close to the value of k of 2.7×10⁻⁹ ml/neutrophil/min for neutrophil killing of 10⁸ C FU/ml S. epidermidis in fibrin gels (Table 1). Using k=2.2-2.3×10⁻⁹ ml/neutrophil/min and g=0.01⁷/min, we calculated CNCs of 7.7 and 7.6×10⁶ neutrophils/ml rabbit dermis 1 and 2 hr, respectively, after E. coli inoculation (Table 2).

By definition, the CNC is the neutrophil concentration which blocks bacterial growth. The E. coli concentration in rabbit dermis peaked between 1 and 2 hr post-E. coli inoculation (FIG. 3a). In this interval the neutrophil concentration in rabbit dermis averaged −7.4×10⁶ neutrophils/ml dermis (i.e., [2.3×10⁶/ml+12.5×10⁶/ml]/2). The very close correspondence of the average neutrophil concentration (i.e., 7.4×10⁶ neutrophils/ml dermis), at 1-2 hr post-E. coli inoculation, and the CNC calculated using Eq. 1 (i.e., 7.7 and 7.6×10⁶ neutrophils/ml dermis), suggests that Eq. 1 accurately estimates neutrophil bactericidal efficiency in rabbit dermis.

The difference between the CNC required for rabbit neutrophils to block growth of −10⁸ CFU E. coli/ml rabbit dermis in vivo, and for human neutrophils to block growth of −10⁸ CFU S. epidennis/ml fibrin gel in vitro (i.e., 7.4-7.7×10⁶ vs. 4.2×10⁶ neutrophils/ml, respectively), is entirely a consequence of differences in growth rates (g) of these bacteria (i.e., 0.01⁷/min vs. 0.01/min, respectively). At equal values 0 f g, the CNCs for these bacteria would be nearly identical (5×10⁶ neutrophils/ml dermis for E. coli vs. 4.2×10⁶/ml fibrin gel for S. epidermidis) despite differences in tissue environments. These results indicate that fibrin gels mimic, and can be used to predict, neutrophil bactericidal activity in vivo.

Discussion

These experiments, and those reported previously, support a quantitative model (Eq. 1) that accurately describes neutrophil bactericidal activity in stirred suspensions (a surrogate for neutrophil bactericidal activity in blood), in fibrin gels (a surrogate for neutrophil bactericidal activity in tissues), and in rabbit dermis in vivo. Li, Y., Karlin, A., Loike, J. D. & Silverstein, S. C. A critical concentration of neutrophils is required for effective bacterial killing in suspension. Proc Natl Acad Sci USA 99, 8289-94. (2002). The model shows that neutrophil bactericidal activity in all three environments depends on the neutrophil concentration and not on the ratio of neutrophils to bacteria.

Eq. 1 precisely models bacterial killing in fibrin gels. Bacteria and neutrophils diffuse freely in stirred suspensions. However, their movements are impeded in fibrin gels. Thus, it was not obvious that Eq. 1, which was derived to describe neutrophil bactericidal activity in stirred suspensions, also would describe neutrophil bactericidal activity in fibrin gels and in tissues. Eq. 1's broad applicability reflects two aspects of k. First, k is independent of neutrophil and bacterial concentration and of bacterial growth rate. Second, variations in other experimental conditions such as IgG and complement concentration, and efficiency of neutrophil migration in three-dimensional matrices, affect the experimentally determined value of bₜ and thereby the value of k (Tables 1 & 2). Li, Y., et al. The bacterial peptide N-fonnyl-Met-Leu-Phe inhibits killing of Staphylococcus epidermidis by human neutrophils in fibrin gels. J Immunol 168, 816-24. (2002).

The Critical Neutrophil Concentration

The finding that the CNC required to block growth of 10⁸ CFU S. epidermidis in fibrin gels (4.2×10⁶ neutrophils/ml), and of 108 CFU E. coli in rabbit dermis (7.7×10⁶ 13 neutrophils/ml), was ˜10-19-fold higher than in stirred suspensions (˜4×10⁵ neutrophils/ml), indicates that the primary reason neutropenia predisposes to sepsis is that the concentration of neutrophils in blood perfusing infected tissues cannot provide enough neutrophils to interdict bacteria that penetrate the body's mucous membranes. Indeed, Koene, et al., reported that sepsis in neutropenic patients correlates more closely with total body mass of neutrophils than with blood neutrophil concentration. Koene, H. R., et al. Clinical value of soluble IgG Fc receptor type III in plasma from patients with chronic idiopathic neutropenia. Blood 91, 3962-6. (1998). Since blood neutrophils comprise less than 5% percent of the body's total neutrophil mass, these findings provide quantitative support for Crosby's suggestion that the tissue neutrophil concentration is the primary determinant of defense against sepsis. Crosby, W. H. How many “polys” are enough? Arch Intern Med 123, 722-3 (1969).

The Neutrophil Extraction Efficiency (NEE)

Using Movat, et al.'s, data for blood flow, blood neutrophil concentration, and neutrophil accumulation in E. coli-infected dermis, we have determined a new parameter which we have termed the neutrophil extraction efficiency (NEE). Movat, H. Z., Cybulsky, M. I., Colditz, I. G., Chan, M. K. & Dinarello, C. A. Acute inflammation in gram-negative infection: endotoxin, interleukin 1, tumor necrosis factor, and neutrophils. FedProc 46, 97-104. (1987); Cybulsky, M. I., Cybulsky, I. J. & Movat, H. Z. Neutropenic responses to intradermal injections of Escherichia coli. Effects on the kinetics of polymorphonuclear leukocyte emigration. Am J Pathol 124, 1-9. (1986); Kopaniak, M. M. & Movat, H. Z. Kinetics of acute inflammation induced by Escherichia coli in rabbits. II. The effect of hyperimmunization, complement depletion, and depletion of leukocytes. Am J Pathol 110, 13-29. (1983). It is the fraction of neutrophils that emigrate from the vasculature into a volume of tissue divided by the total number of neutrophils in blood perfusing that tissue. NEE increased to ˜33% 1-2 hr post-E. coli infection in dermis, and declined steadily thereafter (FIG. 3c).

Kopaniak and Movat reported that E. coli inoculation stimulated similar increases in blood flow in dermis of neutropenic and normal rabbits (FIG. 3c). Kopaniak, M. M. & Movat, H. Z. Kinetics of acute inflammation induced by Escherichia coli in rabbits. II. The effect of hyperimmunization, complement depletion, and depletion of leukocytes. Am J Pathol 110, 13-29. (1983). However, they provided only qualitative data on neutrophil immigration into E. coli-inoculated dermis of neutropenic rabbits. We assumed a blood neutrophil concentration of 5×10⁵/ml and a NEE identical to that in dermis of E. coli-inoculated normal rabbits, and estimated the concentration of neutrophils in the dermis of neutropenic rabbits at various times after E. coli inoculation. Even after 4 hr, the neutrophil concentration in dermis of neutropenic rabbits did not reach the CNC (FIG. 3b). This is consistent with Kopaniak and Movat's 6 finding that E. coli continued to grow at these sites (FIG. 3a). Movat, H. Z., Cybulsky, M. I., Colditz, I. G., Chan, M. K. & Dinarello, C. A. Acute inflammation in gram-negative infection: endotoxin, interleukin 1, tumor necrosis factor, and neutrophils. Fed Proc 46, 97-104. (1987).

NEE, blood neutrophil concentration and blood flow affect the time required for neutrophils reach the CNC in E. coli-infected rabbit dermis. 4

The rate at which neutrophils accumulate in E. coli-infected rabbit dermis determines the extent of bacterial growth at this site (FIG. 3a). Using both experimentally determined and hypothetical values for NEE, blood neutrophil concentration, and blood flow; we calculated the effects of changes in these parameters on the time required for neutrophils to reach the CNC at these sites (Table 3). Kopaniak and Movat reported blood neutrophil concentration averaged −2.5×10⁶/ml during the first 2 hr following E. coli inoculation, while dermal blood flow and NEE increased 4-5-fold and >35 fold, respectively, during this period (FIG. 3c). Cybulsky, M. I., Cybulsky, I. J. & Movat, H. Z. Neutropenic responses to intradermal injections of Escherichia coli. Effects on the kinetics of polymorphonuclear leukocyte emigration. Am J Pathol 124, 1-9. (1986). Thus, the increase in NEE is quantitatively the most important physiological change that leads to increased neutrophil accumulation in infected tissues, making it possible for them to reach the CNC in <2 hr (Table 3).

NEE peaked between 1 and 2 hr after E. coli inoculation, after which it declined rapidly to pre-infection levels (FIG. 3c). Since post-capillary venules regulate neutrophil emigration from the vasculature, they are the cells most likely to be responsible for the observed increases in NEE. Further studies are needed to identify the cellular mechanisms that mediate these changes in NEE. Whatever the mechanisms, they must be specific for neutrophils, because monocyte emigration continued at a steady pace throughout the period of decreasing neutrophil emigration (FIG. 3b). Issekutz, T. B., Issekutz, A. C. & Movat, H. Z. The in vivo quantitation and kinetics of monocyte migration into acute inflammatory tissue. Am J Pathol 103, 47-55. (1981).

Other applications of Eq. 1 and of the CNC concept. Using Eq. 1 it now is possible to determine the CNC required to control bacterial growth in various organs and tissues. Once the CNC has been reached, it may be useful to restrain further neutrophil influx into infected sites. Presumably, this is the reason treatments that reduced neutrophil influx into cerebrospinal fluid of rabbits with pneumococcal meningitis reduced mortality. Tuomanen, Eo I., Saukkonen, K., Sande, S., Cioffe, C. & Wright, S. D. Reduction of inflammation, tissue damage, and mortality in bacterial meningitis in rabbits treated with monoclonal antibodies against adhesion-promoting receptors of leukocytes. J Exp Med 170, 959-69. (1989). Knowledge of the CNC also might be useful in determining the timing and use of antibiotics and/or of granulocyte transfusions in neutropenic patients, and in calculating more precisely the quantity 0 f granulocytes needed to prevent or control bacterial infections in specific organs and tissues in neutropenic patients.

These findings that the neutrophil concentration must exceed the CNC to block bacterial growth, may be applicable to many other situations in biology and medicine. One such situation is immunotherapy of cancer. Tumor-bearing mice and humans often have in their blood cytotoxic lymphocytes that have the capacity to kill autologous tumor cells in vitro, but rarely, if ever, affect these same tumor cells in vivo. While many factors contribute to the inability of cytotoxic lymphocytes to eliminate autologous tumors, the studies reported here suggest that these cytotoxic cells may not accumulate in tumors at a concentration sufficient to kill tumor cells at a rate faster than the tumor cells are growing.

1. A method for treating a biofilm infection in a mammal comprising administering to the mammal a therapeutically effective amount of a composition comprising a complement protein and one or more antibodies which bind to a bacterial, yeast, fungal, carbohydrate or lipid epitope present in the biofilm.

2. A method for treating a biofilm infection in an mammal comprising administering to the mammal a therapeutically effective amount of a composition comprising a complement protein and a conjugate composition, the conjugate composition comprising one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases.

3. The method of claim 2, wherein the protein of the conjugate composition is masked.

4. The method of claim 2, wherein the protein of the conjugate composition is active.

5. The method of claim 1 or 2, wherein the mammal is human.

6. The method of claim 1 or 2, wherein the biofilm is formed on an indwelling device.

7. The method of claim 1 or 2, wherein the biofilm is formed on a prosthetic device.

8. The method of claim 1 or 2, wherein the biofilm is formed on a catheter.

9. The method of claim 1 or 2, wherein the biofilm is formed on tissue.

10. The method of claim 1 or 2, wherein at least one of the antibodies is a monoclonal antibody.

11. The method of claim 10, wherein the monoclonal antibody is a human or humanized monoclonal antibody.

12. The method of claim 1 or 2, wherein the biofilm infection is an S. epidermidis biofllm infection.

13. A method for preventing a biofilm infection in a mammal comprising administering to the mammal a therapeutically effective amount of a composition comprising a complement protein and one or more antibodies which bind to a bacterial, yeast, fungal, carbohydrate or lipid epitope present in the biofilm.

14. A method for preventing a biofilm infection in an mammal comprising administering to the mammal a therapeutically effective amount of a composition comprising a complement protein and a conjugate composition, the conjugate composition comprising one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases.

15. The method of claim 14, wherein the protein of the conjugate composition is masked.

16. The method of claim 14, wherein the protein of the conjugate composition is active.

17. The method of claim 13 or 14, wherein the mammal is human.

18. The method of claim 13 or 14, wherein the biofilm is formed on an indwelling device.

19. The method of claim 13 or 14, wherein the biofilm is formed on a prosthetic device.

20. The method of claim 13 or 14, wherein the biofilm is formed on a catheter.

21. The method of claim 13 or 14, wherein the biofilm is formed on tissue.

22. The method of claim 13 or 14, wherein at least one of the antibodies is a monoclonal antibody.

23. The method of claim 22, wherein the monoclonal antibody is a human or humanized monoclonal antibody.

24. The method of claim 13 or 14, wherein the biofilm infection is an S. epidermidis biofilm infection.

25. A composition for treating a biofilm infection comprising a complement protein and one or more antibodies which bind to a bacterial, yeast, fungal, carbohydrate or lipid epitope present in the biofilm.

26. A composition for treating a biofilm infection comprising a complement protein and a conjugate composition, said conjugate composition comprising: one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases.

27. The composition of claim 26, wherein the protein of the conjugate composition is masked.

28. The composition of claim 26, wherein the protein of the conjugate composition is active.

29. The composition of claim 25 or 26, wherein at least one of the antibodies is a monoclonal antibody.

30. The composition of claim 29, wherein the monoclonal antibody is a human or humanized monoclonal antibody.

31. A kit for use in treating a biofilm infection comprising a complement protein and an antibody which binds to a bacterial, yeast, fungal, carbohydrate or lipid epitope present in the biofilm.

32. A kit for use in treating a biofilm infection comprising a complement protein, and a conjugate composition, said conjugate composition comprising one or more antibodies which bind to a bacterial, yeast, fungal, carbohydrate or lipid epitope present in the biofilm, covalently linked to a protein selected from the group consisting of chemoattractants, chemokines, cytokines, glycosidases or proteases.

33. The kit of claim 32, wherein the protein of the conjugate composition is masked.

34. The kit of claim 32, wherein the protein of the conjugate composition is active.

35. The kit of claim 31 or 32, wherein at least one of the antibodies is a monoclonal antibody.

36. The kit of claim 35, wherein the monoclonal antibody is a human or humanized monoclonal antibody.

37. A method for determining critical neutrophil concentration (CNC) in a pathogen infected tissue comprising determining the concentration of neutrophils accumulated in a volume of the tissue for a period of time after initial infection (NC); determining the growth of the pathogen in the volume of tissue for the period of time after initial infection (PG); calculating the CNC on the basis of the parameters NC and PG by developing an algorithm of determining CNC as a function of NC and PG and applying the values of NC and PG of the tissue under examination to the algorithm.

38. A method for determining neutrophil extraction efficiency of a pathogen infected tissue comprising determining the concentration of neutrophils accumulated in a volume of the tissue for a period of time after initial infection (NC); determining the total number of neutrophils delivered to the volume of the tissue for the period of time after initial infection (NN); calculating the NEE on the basis of the parameters NC and NN by developing an algorithm of determining NEE as a function of NC and NN and applying the values of NC and NN of the tissue under examination to the algorithm.
